(19) United States
(12) Patent Application Publication
(10) Pub. No.: US 2020/0080116 A1
(43) Pub. Date: Mar. 12, 2020

(54) Mesoporous Carbon Modified with Polyethylenimine Catalysis Bisphenol A in Organic Solvent

(71) Applicant: MASSACHUSETTS INSTITUTE OF TECHNOLOGY, Cambridge, MA (US)

(72) Inventor: Lidong Wu, Allston, MA (US)

(73) Assignee: MASSACHUSETTS INSTITUTE OF TECHNOLOGY, Cambridge, MA (US)

(21) Appl. No.: 16/566,170

(22) Filed: Sep. 10, 2019

Related U.S. Application Data
(60) Provisional application No. 62/729,268, filed on Sep. 10, 2018.

(51) Int. Cl. C12P 13/00; B01J 21/18; B01J 31/00; B01J 35/10; C12N 9/02

(52) U.S. Cl. CPC C12P 13/001; B01J 21/18; B01J 31/003; B01J 35/108; C12Y 114/18001; B01J 35/1023; B01J 35/1047; C12N 9/0071; B01J 35/1019

(57) ABSTRACT

An enzyme immobilized on a porous structure can oxidize phenol compounds.

### PRIORITY CLAIM

The application claims priority from U.S. Provisional Patent Application No. 62/729,268, filed Sep. 10, 2018, which is incorporated by reference in its entirety.

### TECHNICAL FIELD

The invention features a composite including an enzyme and methods of oxidizing a substrate with the composite.

### BACKGROUND

Bisphenol A (BPA) is hormone-like chemical that raise concern about its suitability in some consumer products and food containers. In 2012, the United States' Food and Drug Administration (FDA) banned the use of BPA in baby bottles. As of 2014, research and debates are ongoing as to whether BPA should be banned or not. In 2016, an estimated 8 million metric tons of BPA chemical were produced for manufacturing polycarbonate (PC) plastic product. It is one of the highest volume of chemicals produced worldwide.

### SUMMARY

In one aspect, a composite can include a porous carbon carrier, a polymer coating on a surface of the porous carbon carrier, and an enzyme associated with the polymer.

In another aspect, a method of oxidizing a phenol can include suspending a composite including a porous carbon carrier, a polymer coating on a surface of the porous carbon carrier, and an enzyme associated with the polymer in an organic solvent, and exposing the composite to a phenol in the organic solvent. In certain circumstances, the method can include exposing the oxidized phenol to a polyamine to form a polymer.

In certain circumstances, the porous carbon carrier can include carbon nanoparticles, carbon black or a mesoporous carbon. For example, the porous carbon carrier can include a mesoporous carbon.

In certain circumstances, the porous carbon carrier can have a pore-size distribution of about 10 to 50 nm, 15 to 50 nm or 20 to 25 nm.

In certain circumstances, the porous carbon carrier can have a pore-size distribution of about 20 to 25 nm.

In certain circumstances, the porous carbon carrier can have a specific surface area of between 300 and 800 m² g⁻¹, between 400 and 700 m² g⁻¹, or between 500 and 600 m² g⁻¹. In certain circumstances, the porous carbon carrier can have a pore volume of between 1.5 and 2.5 cm³ g⁻¹.

In certain circumstances, the polymer can include a plurality of amino groups.

In certain circumstances, the polymer can include polyethyleneimine, a polyethylene glycol, a polyacrylate, triethylaminoethyl cellulose, diethylaminoethyl cellulose, cellulose, carboxymethyl cellulose, bovine serum albumin (BSA), or a lysozyme. In certain circumstances, the polymer can include polyethyleneimine or a triethylaminoethyl cellulose.

In certain circumstances, the enzyme can be a tyrosinase.

In certain circumstances, the organic solvent can include an aromatic solvent, for example, benzene or toluene.

In certain circumstances, the phenol can include bisphenol A.

In certain circumstances, the porous carbon carrier:polymer can have a ratio of between 4:1 and 1:4, between 2:1 and 1:2 or around 1:1 (wt/wt).

Other aspects, embodiments, and features will be apparent from the following description, the drawings, and the claims.

### BRIEF DESCRIPTION OF THE DRAWINGS

FIG. 1 is a set of SEM micrographs depicting mesoporous carbon, mesoporous carbon with PEI and mesoporous carbon with PEI and tyrosinase.

FIG. 2 is a set of TEM images of mesoporous carbon, mesoporous carbon with PEI and mesoporous carbon with PEI and tyrosinase.

FIG. 3 is a graph depicting Vₘₐₓ values of tyrosinase molecular immobilization onto MPC/PEI, MPC/PAL, MPC/PEG and MPC catalyzed oxidations of p-cresol as substrate in toluene. P-cresol concentration for these reactions is 50 mmol L⁻¹. Error bar means standard deviation.

FIG. 4 is a graph depicting Vₘₐₓ values of tyrosinase molecular immobilization onto MPC/PEI catalyzed oxidations of p-cresol as substrate in different water content of toluene. P-cresol concentration for these reactions is 50 mmol L−1. Error bar means standard deviation.

FIG. 5 depicts HPLC-MS analysis of bisphenol A product after mixing with immobilized enzyme for 15 minutes in toluene with 6% water content.

FIG. 6 depicts oxidation of bisphenol A by immobilized tyrosinase under oxygen.

FIG. 7 depicts reaction of amines with 4,4′-(propane-2,2-diyl)bis(cyclohexa-3,5-diene-1,2-dione) via Michael-type addition or Schiff base reaction.

FIG. 8 depicts a chemical structure of bisphenol A.

FIG. 9 are graphs. Panel A shows Vₒ values of tyrosinase immobilized on mesoporous carbon and PEI coated mesoporous carbon (the weight ratio of mesoporous carbon:PEI is 1:1) with different water content. Panel B shows Vₒ values of tyrosinase immobilization onto PEI/mesoporous carbon (the weight ratio mesoporous carbon:PEI are 4:1, 2:1, 1:1 and 1:2, respectively) catalyzed oxidations of p-cresol as substrate in toluene. P-cresol concentration for these reactions is 50 mmol L⁻¹. Error bar means standard deviation. Vₒ is the amount of the oxidation product increased per min and per mg immobilized enzyme, measured by the absorption spectra. Inset: Chemical structure of p-cresol.

FIG. 10 depicts micrographs and a graph of pore size. SEM images of (panel A) mesoporous carbon, (panel B) mesoporous carbon with PEI, and (panel C) mesoporous carbon with PEI and tyrosinase. TEM images of (panel D) Mesoporous carbon, (panel E) enlarged mesoporous carbon, (panel F) mesoporous carbon with PEI, and (panel G) mesoporous carbon with PEI and tyrosinase. Panel H shows the corresponding BJH pore size distribution of mesoporous carbon, PEI/mesoporous carbon and PEI/mesoporous carbon with tyrosinase.

FIG. 11 depicts the process of PEI coating mesoporous carbon to immobilize tyrosinase (panel A), PEG coating mesoporous carbon to immobilize tyrosinase (panel B) and PAL coating mesoporous carbon to immobilize tyrosinase (panel C). The brown dots represent the coating materials in the scheme. (panel D) the photograph of powder of PEI coating mesoporous carbon, (panel E) the photograph of powder of PEG coating mesoporous carbon and (panel F) the photograph of powder of PAL coating mesoporous carbon.

FIG. 12 depicts HPLC-MS analysis of BPA product after mixing with the immobilized-tyrosinase for 60 minutes in toluene with 6% water content (panel A), and for 10 hours in toluene with 6% water content (panel B).

FIG. 13 depicts a possible reaction pathways for bisphenol A oxidation by immobilized-tyrosinase under oxygen.

FIG. 14 depicts reaction of amines with 4,4′-(propane-2,2-diyl)bis(cyclohexa-3,5-diene-1,2-dione) via Michael-type addition or Schiff base reaction.

FIG. 15 depicts images. TEM image of poly(amino-quinone) polymers formed by hexamethylenediamine and 4,4′-(1-methylethylidene)bis(1,2-benzoquinone) (panel A), SEM images this polymer (panel B), the photograph of the reaction product poly(amino-quinone) polymers, the dilution of poly(amino-quinone) polymers in the upper vial and the poly(amino-quinone) polymers deposited on the bottom of glass bottle (panel C), the reaction of hexamethylenediamine with 4,4′-(propane-2,2-diyl)bis(cyclohexa-3,5-diene-1,2-dione) through Schiff Base Reaction (panel D).

FIG. 16 depicts a TEM image of poly(amino-quinone) polymers formed by trilysine and 4,4′-(1-methylethylidene)bis(1,2-benzoquinone) (panel A) and the reaction mechanism of trilysine and 4,4′-(1-methylethylidene)bis(1,2-benzoquinone) (panel B).

FIG. 17 depicts oxidation of p-cresol by tyrosinase immobilized on mesoporous carbon or coated mesoporous carbon.

FIG. 18 depicts comparison of the oxidation BPA by enzyme biocatalysis method with other oxidation methods.

FIG. 19 depicts images. The SEM of (panel A) triethylaminoethyl cellulose (TEAE-Cellulose) and (panel B) TEAE-Cellulose with tyrosinase; the TEM of (panel C) triethylaminoethyl cellulose (TEAE-Cellulose) and (panel D) TEAE-Cellulose with tyrosinase.

FIG. 20 depicts SEM images of (panel A) Cellulose, (panel B) CM-Cellulose, (panel C) Pluronic F68, (panel D) Poly(styrene-co-divinylbenzene) and (panel E) Glass beads. After immobilization tyrosinase onto the surface of materials, Cellulose (panel H), CM-Cellulose (panel I) and Pluronic F68 (panel K) morphologies totally changed and transform into another morphologies, and all of the materials surface (panel L) became rough.

FIG. 21 depicts schematic diagrams describing the mechanism of tyrosinase immobilization onto (panel A) CM-Cellulose; (panel B) Cellulose; and (Panel C) TEAE-Cellulose.

FIG. 22 is a graph depicting Vₒ values of tyrosinase molecular immobilization onto CM-Cellulose, Cellulose, TEAE-Cellulose, Pluronic F68, Poly(styrene-co-divinylbenzene) and Glass beads catalyzed oxidations of p-cresol as substrate in chloroform. P-cresol concentration for these reactions is 50 mmol L−1. Error bar means standard deviation.

FIG. 23 depicts activity of tyrosinase immobilization onto TEAE-Cellulose monitored in different solvent (i.e. CH₂Cl₂, CCl₄ and toluene).

FIG. 24 depicts bioactivity of tyrosinase was monitored for different substituent sites in different solvent (i.e. CH₂Cl₂, CCl₄ and toluene).

FIG. 25 depicts bioactivity of tyrosinase was detected for six kinds of para-substituent of phenol in toluene.

FIG. 26 shows graphs that depict (panel A) The half-life of the immobilized tyrosinase vs the log P values of the organic solvent, and (panel B) The half-life of the immobilized tyrosinase vs the relative polarity values of the organic solvent. The organic solvent from left to right is methanol, ethanol, acetonitrile, acetone, methyl acetate, tetrahydrofuran, methyl t-butyl ether, toluene and hexane.

FIG. 27 depicts coating materials and activity of composites disclosed herein.

FIG. 28 is a graph depicting stability of immobilized tyrosinase showing that it maintains over 80% activity in toluene at room temperature for one week.

### DETAILED DESCRIPTION

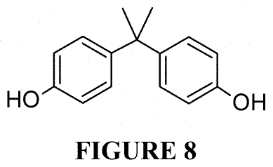

Bisphenol A (BPA; FIG. 8) is a widely used chemical but toxic, and its biodegradation in aqueous environment is hard due to its near insolubility in water. While the enzyme tyrosinase can oxidize BPA in organic solvents, it does so only very slowly. In the present study, we have found that in toluene the catalytic activity of tyrosinase deposited onto coated mesoporous carbon is markedly enhanced when the support is pre-coated with polyethyleneimine. The resultant enzymatically formed o-quinone is both readily recoverable and potentially useful monomers. As a particular example, the o-quinone readily react with diamine in toluene to form poly(amino-quinone) polymers which are suitable for anticorrosion, energy storage or biosensor applications.

Bisphenol A (BPA) is one of the highest volume industrial chemicals used as a starting material to produce polycarbonate and epoxy resins. But BPA is identified as a xenoestrogen, as a result in 2012 was banned in the United States for use in baby bottles due to its reproductive and developmental toxicity. Therefore, it can be desirable to convert it into o-quinones which easily react with nucleophiles to form polymers for anticorrosion, energy storage, and biosensor, et. al. See, for example, Le, H. H.; Carlson, E. M.; Chua, J. P.; Belcher, S. M. Toxicol Lett 2008, 176, (2), 149-156; Patisaul, H. B.; Todd, K. L.; Mickens, J. A.; Adewale, H. B. Neurotoxicology 2009, 30, (3), 350-357; Gear, R. B.; Belcher, S. M. Sci Rep-Uk 2017, 7, (856), 1-8; Chen, S.; Zhang, J.; Chen, Y.; Zhao, S.; Chen, M.; Li, X.; Maitz, M. F.; Wang, J.; Huang, N. ACS Appl Mater Interfaces 2015, 7, (44), 24510-24522; Pham, M. C.; Hubert, S.; Piro, B.; Maurel, F.; Le Dao, H.; Takenouti, H. Synthetic Met 2004, 140, (2-3), 183-197; Liang, Y. L.; Jing, Y.; Gheytani, S.; Lee, K. Y.; Liu, P.; Facchetti, A.; Yao, Y. Nat Mater 2017, 16, (8), 841-848; Navarro-Suarez, A. M.; Carretero-Gonzalez, J.; Rojo, T.; Armand, M. J Mater Chem A 2017, 5, (44), 23292-23298; Arai, G.; Shoji, K.; Yasumori, I. J Electroanal Chem 2006, 591, (1), 1-6; and Martin, M.; Orive, A. G.; Lorenzo-Luis, P.; Creus, A. H.; Gonzalez-Mora, J. L.; Salazar, P. Chemphyschem 2014, 15, (17), 3742-3752, each of which is incorporated by reference in its entirety. In previous research, Fremy's salt was used for oxidation of phenolic compounds. See, for example, Hans Zimmer; David C. Lankin; Horgan, S. W. Chemical Reviews 1970, 71, (2), 229-247, which is incorporated by reference in its entirety. However, this salt is unstable, releases flammable gases, and oxidizes extensive aromatic amines and phenols into quinones unselectively. Regio- and chemo-selective oxidation of BPA under mild conditions has been a longstanding goal. See, for example, Esguerra, K. V. N.; Lumb, J. P. Angew Chem Int Edit 2018, 57, (6), 1514-1518, which is incorporated by reference in its entirety.

The enzyme tyrosinase (also called polyphenol oxidase; EC 1.14.18.1) may overcome these challenges and be a good candidate for regio- and chemo-selective oxidation of BPA under mild condition. Tyrosinase catalyzes the hydroxylation of phenols to catechols and subsequent dehydrogenation to o-quinones. Dopamine and L-tyrosine are easily oxidized by oxygen under tyrosinase catalysis in aqueous solution. BPA has poor solubility in water but is readily soluble in organic solvent, for example, it is over an order of magnitude more soluble in toluene than in water. Meanwhile, there is an extra benefit that the co-substrate oxygen has nearly 40-fold higher solubility in toluene than in water. While tyrosinase has been deposited onto glass beads to catalyze oxidation of various phenols in organic solvents, the resultant enzymatic activity toward BPA is negligible, in part due to a low available surface area which severely restricts the loading capacity of the carrier. Therefore, it is surprisingly found that mesoporous carbon as an immobilization support to enhance tyrosinase activity toward BPA in organic solvents.

A composite can include a porous carbon carrier, a polymer coating on a surface of the porous carbon carrier, and an enzyme associated with the polymer. The composite can be used to oxidize a phenol in an organic solvent. The oxidation can take place when the enzyme is a tyrosinase, which can be an enzyme with the ability to oxidize tyrosine. The method of oxidizing a phenol can include suspending a composite including a porous carbon carrier, a polymer coating on a surface of the porous carbon carrier, and an enzyme associated with the polymer in an organic solvent, and exposing the composite to a phenol in the organic solvent.

When the oxidation takes place in the presence of a polyamine, the oxidized phenol can form an imine or addition product. If the phenol is a polyphenol, such as BPA, the oxidized phenol can react with a polyamine to form a polymer. The polyamine can be a triamine or a diamine, for example, diamino ethane, a polylysine, or similar compound.

The porous carbon carrier can include carbon nanoparticles, carbon black or a mesoporous carbon. For example, the porous carbon carrier can include a mesoporous carbon. The porous carbon carrier can have a pore-size distribution of about 10 to 50 nm, 15 to 50 nm or 20 to 25 nm, for example, about 22 nm. The porous carbon carrier can have a specific surface area of between 300 and 800 m² g⁻¹, between 400 and 700 m² g⁻¹, or between 500 and 600 m² g⁻¹. The porous carbon carrier can have a pore volume of between 1.5 and 2.5 cm³ g⁻¹.

The polymer can be a polycation, a polyanion, or a zwitterionic polymer. The polymer can include a plurality of amino groups. In certain circumstances, the polymer can include polyethyleneimine, a polyethylene glycol, a polyacrylate, triethylaminoethyl cellulose, diethylaminoethyl cellulose, cellulose, carboxymethyl cellulose, bovine serum albumin (BSA), or a lysozyme. In certain circumstances, the polymer is a polycation and can include polyethyleneimine or a triethylaminoethyl cellulose.

The polymer can coat a surface of the porous carbon carrier. In certain circumstances, the porous carbon carrier:polymer can have a ratio of between 4:1 and 1:4, between 2:1 and 1:2 or around 1:1 (wt/wt). The coating can cover at least 40%, at least 50%, at least 60%, at least 70%, at least 80% or at least 90% of the surface of the porous carbon carrier.

The organic solvent can include an aromatic solvent, for example, benzene or toluene.

In certain circumstances, the phenol can include bisphenol A.

The mesoporous carbon substrate has large surface area, high pore volume and well-controlled pore size distribution allowing for high loading efficiency of the tyrosinase. Furthermore, it is a stable platform with high corrosion resistance and good thermal and mechanical properties. But there is a certain degree of conformational change of enzyme during the enzyme adsorption onto the carbon materials, which resulted in loss of biological activity. The combination of polymers with mesoporous carbon might change its surface properties and create a biocompatible nanocomposite. In this work, several types of polymers are used for coating the surface of mesoporous carbon. Compared with the bare carrier, polyethyleneimine (PEI) coated mesoporous carbon as a tyrosinase immobilization platform improves over an order of magnitude higher enzymatic activity than just the mesoporous carbon. BPA is successfully converted into 4,4′-(1-methylethylidene)bis(1,2-benzoquinone) which could be used as a monomer to react with diamine to form poly(amino-quinone) polymers for applications in anticorrosion, energy storage and biosensors.

Tyrosinase is a copper containing enzyme which is ubiquitous from bacteria to mammals. See, for example, Sanchezferrer A, Rodriguezlopez J N, Garciacanovas F, Garciacarmona F. Tyrosinase—a Comprehensive Review of Its Mechanism. Bba-Protein Struct. M. 1995; 1247:1-11 and Jamshidzadeh A, Shokri Y, Ahmadi N, Mohamadi N, Sharififar F. Quercus infectoria and Terminalia chebula decrease melanin content and tyrosinase activity in B16/F10 cell lines. J. Pharm. Pharmacogn. R. 2017; 5:270-7, each of which is incorporated by reference in its entirety. It is well-known that tyrosinase catalyzes hydroxylation of phenols with oxygen to catechols and subsequent dehydrogenation to o-quinones. Subsequently o-quinones are highly reactive moleculars which play important role in the adhesion of mussel-inspired chemistry, undergo non-enzymatic secondary reactions to form brown complex polymer known as melanin in water and readily interact with nucleophiles (e.g. NH₂ or SH) to cross-link with protein functional groups. See, for example, Bizzarri B M, Martini A, Serafini F, Aversa D, Piccinino D, Botta L, et al. Tyrosinase mediated oxidative functionalization in the synthesis of DOPA-derived peptidomimetics with anti-Parkinson activity. Rsc Adv. 2017; 7:20502-9; Le Thi P, Lee Y, Nguyen D H, Park K D. In situ forming gelatin hydrogels by dual-enzymatic cross-linking for enhanced tissue adhesiveness. J Mater Chem B. 2017; 5:757-64; and Isaschar-Ovdat S, Fishman A. Mechanistic insights into tyrosinase-mediated crosslinking of soy glycinin derived peptides. Food Chem. 2017; 232:587-94, each of which is incorporated by reference in its entirety. But these active o-quinones in water causes rapid polymerization to polyaromatic pigment which inactivates the tyrosinase. See, for example, Kazandjian R Z, Klibanov A M. Regioselective Oxidation of Phenols Catalyzed by Polyphenol Oxidase in Chloroform. J Am Chem Soc. 1985; 107:5448-50; and Atlow S C, Bonadonnaaparo L, Klibanov A M. Dephenolization of Industrial Wastewaters Catalyzed by Polyphenol Oxidase. Biotechnol Bioeng. 1984; 26:599-603, each of which is incorporated by reference in its entirety.

To avoid the o-quinone polymerization in aqueous solution, tyrosinase catalyze phenol derivative in hydrophobic organic solvent can solve the above problem. The immobilization material is the key of enzyme catalysis in vitro. See, for example, Gascon V, Castro-Miguel E, Diaz-Garcia M, Blanco R M, Sanchez-Sanchez M. In situ and post-synthesis immobilization of enzymes on nanocrystalline MOF platforms to yield active biocatalysts. J Chem Technol Biot. 2017; 92:2583-93; Lian X Z, Fang Y, Joseph E, Wang Q, Li J L, Banerjee S, et al. Enzyme-MOF (metal-organic framework) composites. Chem Soc Rev. 2017; 46:3386-401; and Liu Y, Turner A P F, Zhao M J, Mak W C. Processable enzyme-hybrid conductive polymer composites for electrochemical biosensing. Biosens Bioelectron. 2018; 100:374-81, each of which is incorporated by reference in its entirety. Carbon material (e.g. graphene, multiwall nanotubes, mesoporous carbon, fullerene) was one of the hottest materials among these years which possess many attractive properties for use as immobilized enzyme support, such as reasonable price, high specific surface area, good electric conductivity, and excellent electrochemical and mechanical stability. See, for example, Cho Y K, Bailey J E. Immobilization of Enzymes on Activated Carbon—Properties of Immobilized Glucoamylase, Glucose Oxidase, and Gluconolactonase. Biotechnol Bioeng. 1978; 20:1651-65; Laurent C, Dinh T M, Barthelemy M C, Chevallier G, Weibel A. Mesoporous binder-free monoliths of few-walled carbon nanotubes by spark plasma sintering. J Mater Sci. 2018; 53:3225-38; Ebrahimi M, Rosei F. MATERIALS SCIENCE Organic analogues of graphene. Nature. 2017; 542:423-4; and Lee M L. MATERIALS SCIENCE Crystals aligned through graphene. Nature. 2017; 544:301-2, each of which is incorporated by reference in its entirety. But until now there is no research to assess the activity of immobilized tyrosinase with carbon materials, lots of research focus on immobilizing tyrosinase onto carbon materials to detect phenolic compounds by electrochemical or fluorometric method. See, for example, Zhao X E, Lei C H, Wang Y H, Qu F, Zhu S Y, Wang H, et al. A fluorometric assay for tyrosinase activity and its inhibitor screening based on graphene quantum dots. Rsc Adv. 2016; 6:72670-5; Wu L D, Deng D H, Jin J, Lu X B, Chen J P. Nano-graphene-based tyrosinase biosensor for rapid detection of bisphenol A. Biosens Bioelectron. 2012; 35:193-9; Apetrei I M, Rodriguez-Mendez M L, Apetrei C, de Saja J A. Enzyme sensor based on carbon nanotubes/cobalt(II) phthalocyanine and tyrosinase used in pharmaceutical analysis. Sensor Actuat B-Chem. 2013; 177:138-44; Kochana J, Wapiennik K, Knihnicki P, Pollap A, Janus P, Oszajca M, et al. Mesoporous carbon-containing voltammetric biosensor for determination of tyramine in food products. Anal Bioanal Chem. 2016; 408:5199-210, each of which is incorporated by reference in its entirety. Why all of them did not assess the activity of tyrosinase immobilized onto carbon material, only used it as a biosensor. The reason was that the high conductivity, large surface area and strong physical absorption of carbon material could be the advantage for immobilized tyrosinase as a biosensor but its strong physical absorption could absorb most of the substrate and product from aqueous solution to carbon materials. See, for example, Li Z H, Wang W B, Cao H B, Zhang Q, Zhou X Y, Wang D B, et al. Boron Doped ZIF-67@Graphene Derived Carbon Electrocatalyst for Highly Efficient Enzyme-Free Hydrogen Peroxide Biosensor. Adv Mater Technol-Us. 2017; 2; Galdino N M, Brehm G S, Bussamara R, Goncalves W D G, Abarca G, Scholten J D. Sputtering deposition of gold nanoparticles onto graphene oxide functionalized with ionic liquids: biosensor materials for cholesterol detection. J Mater Chem B. 2017; 5:9482-6; Fu C X, Wang Z F, Liu J Y, Jiang H B, Li G M, Zhi C Y. Large scale fabrication of graphene for oil and organic solvent absorption. Prog Nat Sci-Mater. 2016; 26:319-23; and Sun H X, Zhu Z Q, Liang W D, Yang B P, Qin X J, Zhao X H, et al. Reduced graphene oxide-coated cottons for selective absorption of organic solvents and oils from water. Rsc Adv. 2014; 4:30587-91, each of which is incorporated by reference in its entirety.

Mesoporous carbon was selected to immobilize tyrosinase and assessed the immobilized tyrosinase in toluene. This method conquered the strong physical absorption of mesoporous carbon and could continually obtain quinones without polymerization. Biocompatible polymer (e.g. PEI, PEG and PAL) used for precoating mesoporous carbon to get a good tyrosinase immobilized platform. Bisphenol A had stronger steric hindrance than p-cresol and its oxidation product might be 4,4′-(propane-2,2-diyl)bis(cyclohexa-3,5-diene-1,2-dione) with mirror symmetry structure which could be a cross-linker between protein functional groups and polymer. Finally, bisphenol A regioselectively converted into 4,4′-(propane-2,2-diyl)bis(cyclohexa-3,5-diene-1,2-dione) and bisphenol A product could form polymer in organic solvent.

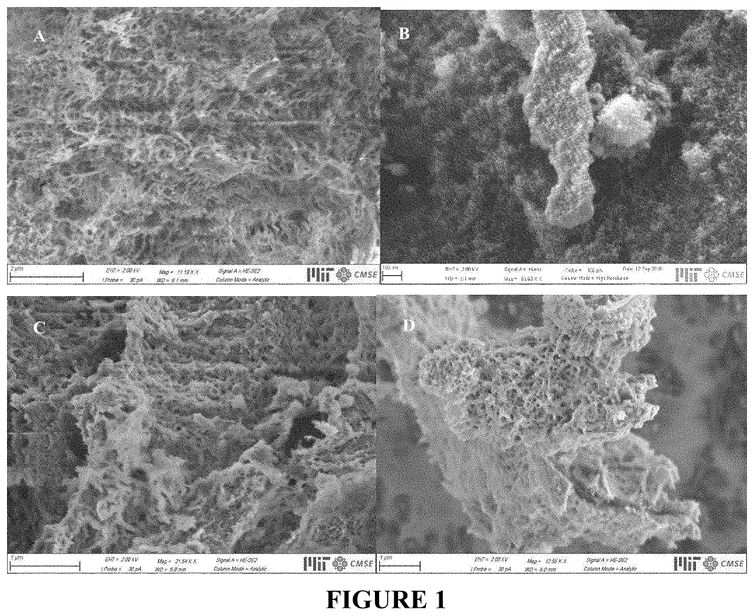

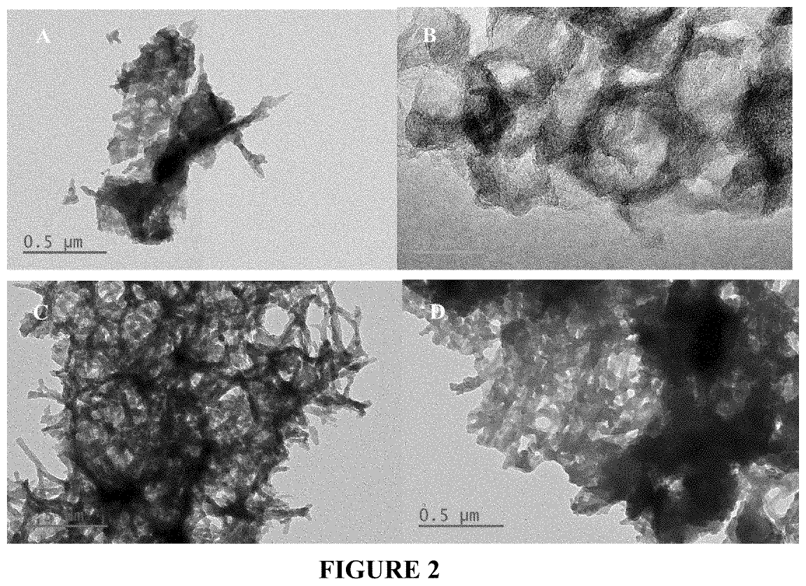

Physical characterization of mesoporous carbon/polyethyleneimine (MPC/PEI) and tyrosinase immobilization onto MPC/PEI. FIG. 1, panel A (SEM image) displayed the morphological features of MPC, and FIG. 1, panel B (the enlarge view of FIG. 1, panel A) displayed the ordered pores morphology of MPC. FIG. 2, panel A and 2, panel B (the enlarge view of FIG. 2, panel A) displayed uniformly distributed spherical mesopores of MPC by TEM, and its surface structure was consistent with SEM images. After precoating of PEI, as shown in FIG. 1, panel C, there was a thin layer on the surface of MPC. Then, after immobilization of tyrosinase molecules in the MPC/PEI (FIG. 1, panel D), the MPC/PEI surface became rougher than the MPC/PEI. The TEM images (FIG. 2, panel C and 2, panel D) were consistent with SEM results. After immobilization of tyrosinase, these performs of these materials were assessed by catalysis ability of their immobilization tyrosinase. TEM images showed A) MPC with tyrosinase, B) MPC/PAL, C) MPC/PAL with tyrosinase, D) MPC/PEG and E) MPC/PEG with tyrosinase with tyrosinase. After tyrosinase immobilization onto the surface of materials, all of the surface showed rougher than the original one. These alterations showed that the tyrosinase have been immobilized onto the surface of materials.

The pore-size distribution of MPC was mainly in the range of 22 nm. Its specific surface area and pore volume were 582 m² g⁻¹ and 2.1 cm³ g⁻¹, respectively. After pre-coating PEI on MPC surface, its surface area and pore volume decrease to 505 m² g⁻¹ and 1.9 cm³ g⁻¹, respectively. This demonstrated that MPC surface have been coated by PEI. After tyrosinase immobilization onto MPC/PEI surface, its surface area and pore volume decrease to 200 m² g⁻¹ and 1.3 cm³ g⁻¹, respectively. When PAL or PEG coated the surface of MPC, the surface area and pore volume decrease to 440 m² g⁻¹ or 525 m² g⁻¹ and 1.7 cm³ g⁻¹ or 1.95 cm³ g⁻¹, respectively. As tyrosinase immobilized onto the surface of MPC/PAL or MPC/PEG, their surface area and pore volume continue to decrease to 175 m² g⁻¹ or 205 m² g⁻¹ and 1.15 cm³ g⁻¹ or 1.4 cm³ g⁻¹, respectively. The results proved that polymer coated the surface of MPC and tyrosinase immobilized onto the nanocomposite.

Effects of Immobilization Material on Tyrosinase Biocatalysis Ability.

MPC has intriguing physical and chemical properties, such as high specific surface area, large pore volume, tunable pore size, good electric conductivity, and excellent electrochemical and mechanical stability. Although there are abundant of research using carbon material (mesoporous carbon, graphene and Multi-walled nanotubes) to immobilize enzyme as biosensor platform, there is no research using them as an enzyme immobilization platform to catalyze phenol derivative reaction in solution until now. Because there is a technique barrier, that carbon material has strong physical absorption ability to grab almost all of phenol derivative from aqueous or air to carbon material. As shown herein, mesoporous carbon can adsorb 90% p-cresol or 87% bisphenol A from their aqueous solution. This is overcome by dissolving p-cresol or bisphenol A in toluene. This method could improve bisphenol A solubility from 1 mmol L⁻¹ in water to 10 mmol L⁻¹ in toluene, and p-cresol could be quantitatively converted to stable o-quinones in toluene to avoid instability of o-quinones in water causing rapid polymerization to polyaromatic pigments which inactive the enzyme. MPC only can adsorb 4% p-cresol or 3% bisphenol A from toluene solution. So MPC used as immobilization platform for tyrosinase and the activity of immobilized tyrosinase was tested in toluene, unfortunately, the activity of tyrosinase immobilization onto MPC was not detectable by UV-Vis spectroscopy.

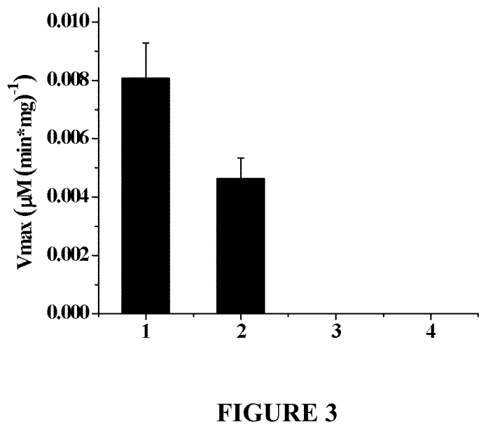

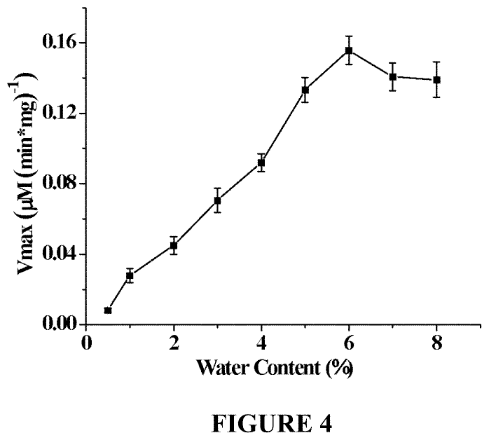

How to improve the activity of tyrosinase immobilization onto MPC might ignite the new engine for carbon material in biocatalysis. Three different polymers were used as precoating material for MPC. After MPC coated with polymer, the activity of immobilized tyrosinase was tested. FIG. 3 showed that the activities of tyrosinase/MPC/PEI, tyrosinase/MPC/PAL and tyrosinase/MPC/PEG were 0.0081, 0.004 and 0, respectively. PEI was the best coated polymer for MPC. Subsequently, the effect of water content on enzymatic reaction rate in toluene were investigated. As shown in FIG. 4, the higher the water content before adding 6% water, the greater the enzymatic activity. A qualitatively similar trend was observed as reported. See, for example, Zaks A, Klibanov A M. The Effect of Water on Enzyme Action in Organic Media. J Biol Chem. 1988; 263:8017-21, which is incorporated by reference in its entirety. The activities of tyrosinase/MPC/PEI with 6% water content in toluene achieved 0.156. After optimizing the water content in toluene, the mass ratio between MPC and PEI as enzyme immobilization platform was investigated. The results showed that the ratio between MPC and PEI (1:1) was the best platform for enzyme immobilization.

Oxidation of Bisphenol a Using the Immobilized Tyrosinase.

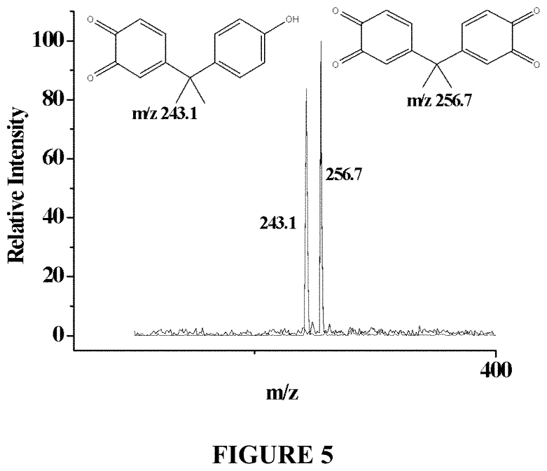

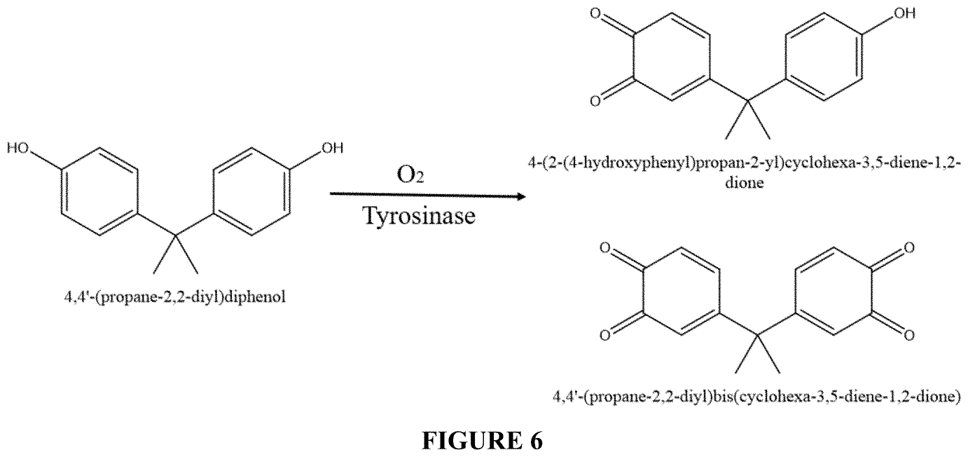

Bisphenol A is a phenol derivative (endocrine disrupting chemical) with stronger steric hindrance than p-cresol. And Endo et al. reported that bisphenol A was not oxidized in the presence of tyrosinase from mushroom, even p-octylphenol could be oxidized by tyrosinase. See, for example, Endo Y, Xuan Y J, Fujimoto K. The oxidation of p-octylphenol by mushroom tyrosinase. Nippon Nogeik Kaishi. 2000; 74:1337-41, which is incorporated by reference in its entirety. After optimizing the best platform for tyrosinase immobilization, the immobilized tyrosinase used for oxidation of bisphenol A in toluene. The activity of tyrosinase/MPC/PEI for bisphenol A (10 mmol L⁻¹) in toluene with 6% water was 0.039. The oxidation of bisphenol A by immobilized tyrosinase, a radical oxidant was done as the next trial (FIG. 6). HPLC-MS analysis (FIG. 5) identified that after 60 minutes of reaction catalyzed by immobilized tyrosinase, there was 4-(2-(4-hydroxyphenyl)propan-2-yl)cyclohexa-3,5-diene-1,2-dione and 4,4′-(propane-2,2-diyl)bis(cyclohexa-3,5-diene-1,2-dione) as oxidation products. The ratio between 4-(2-(4-hydroxyphenyl)propan-2-yl)cyclohexa-3,5-diene-1,2-dione and 4,4′-(propane-2,2-diyl)bis(cyclohexa-3,5-diene-1,2-dione) was almost 1:1. After 10 hours, most of the oxidation product was 4,4′-(propane-2,2-diyl)bis(cyclohexa-3,5-diene-1,2-dione). It should be stressed that the quinone initially formed readily polymerizes in the aqueous solution to inactive the tyrosinase and bisphenol A in toluene vs water are a 10-fold higher solubility. But in this system, we could not compare the activity of tyrosinase/MPC/PEI due to high physical absorption of MPC (90% p-cresol or 87% bisphenol A absorb by MPC in water).

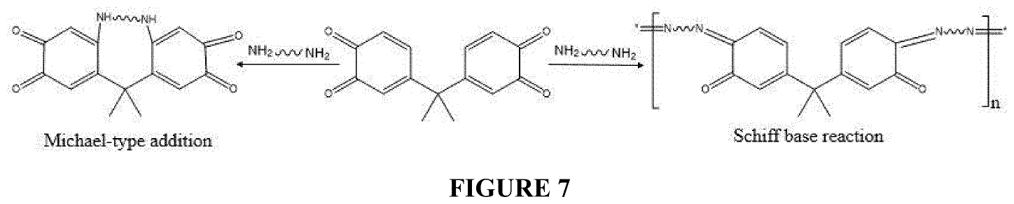

The formed 4,4′-(propane-2,2-diyl)bis(cyclohexa-3,5-diene-1,2-dione) has four carbonyl groups which may further react with a variety of nucleophiles in various pathways to form crosslinks. A well-known nucleophile is the amine that may react with o-quinones to form adducts either by Michael addition or Schiff base reaction (FIG. 7). Therefore, in this study, 4,4′-(propane-2,2-diyl)bis(cyclohexa-3,5-diene-1,2-dione) and hexamethylenediamine was used to study the crosslinking chemistry. After adding hexamethylenediamine into 4,4′-(propane-2,2-diyl)bis(cyclohexa-3,5-diene-1,2-dione) toluene solution, the polymer are formed. And trilysine aqueous solution mixed with 4,4′-(propane-2,2-diyl)bis(cyclohexa-3,5-diene-1,2-dione) solution under stirring, it formed tree branch network. Based on the mirror symmetry of 4,4′-(propane-2,2-diyl)bis(cyclohexa-3,5-diene-1,2-dione) structure, it could be a potential monomer for polymer or metal-organic framework.

As described herein, bisphenol A regioselective oxidation using tyrosinase was investigated in toluene solution with oxygen and little water. Mesoporous carbon coated with PEI which had large surface area, large pore volume and strong affinity with tyrosinase was made as an attractive platform for enzyme immobilization. This conquered the hypothesis which proposed by Endo “Endo et al. reported that bisphenol A was not oxidized in the presence of tyrosinase from mushroom.” In the system described here, the enzyme activity after immobilization onto carbon material could be assessed in organic solution which avoid adsorbing the substrate and product onto the surface of carbon material. The stable product of bisphenol A could be used as a potential monomer for polymer or metal-organic framework.

In summary, bisphenol A regioselective oxidation using tyrosinase was investigated in toluene solution with oxygen and little water. Due to the low activity of tyrosinase in organic solution, mesoporous carbon (MPC) coated with Polyethyleneimine (PEI) which had large surface area, large pore volume and strong affinity with tyrosinase was used as a platform for enzyme immobilization. Compared with MPC, polyethylene glycol (PEG) coated MPC and polyacrylate (PAL) coated MPC, PEI coated MPC was the best immobilization platform for tyrosinase. the activities of tyrosinase/MPC/PEI, tyrosinase/MPC/PAL, tyrosinase/MPC/PEG and tyrosinase/MPC for p-cresol were 0.0081, 0.004, 0 and 0, respectively. After optimizing experiment condition, the activity of tyrosinase/MPC/PEI for bisphenol A (10 mmol L⁻¹) in toluene with 6% water was 0.039. The stable product of bisphenol A could be used as a potential monomer for polymer or metal-organic framework. The reaction system avoided adsorbing the substrate and product onto the surface of carbon material and assessed the enzyme activity after immobilization onto the carbon material, which might ignite the new engine for carbon material as enzyme immobilization platform.

Effects of the Immobilization Material on the Tyrosinase Biocatalysis Ability.

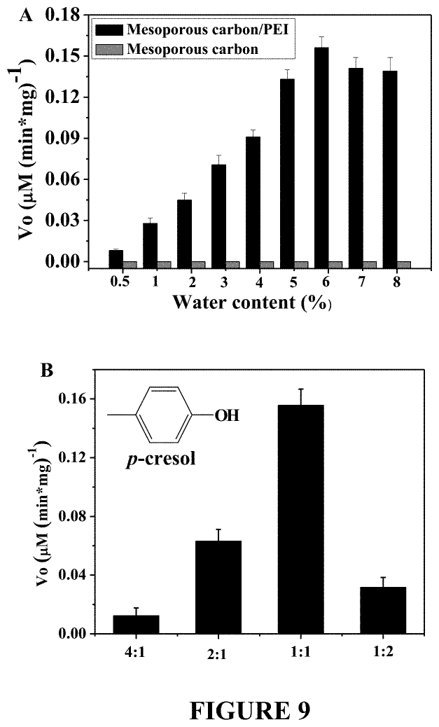

To study the tyrosinase catalysis in organic solvent, p-cresol was employed as a model substrate, which is more easily oxidized than BPA. Its chemical structure is much simpler than BPA, as shown in the inset of FIG. 9. The tyrosinase-catalyzed oxidation reaction was investigated by monitoring the oxidation product of p-cresol. No significant formation of the enzymatic oxidation product was detected with 10 mg mL⁻¹ free tyrosinase suspended in toluene. It is likely due to the decreased conformational mobility of the enzyme structure and the free form of tyrosinase inactivation in organic solvent. The use of immobilized enzyme instead of free form represents the most common method for improving enzyme stability toward organic solvents. The immobilized enzyme has more rigid conformation, which prevents unfolding of the enzyme and malformation of its active site. First, the tyrosinase was precipitated onto the glass beads. The enzymatic oxidation rate (defined as V.) is the amount of the oxidation product increased per min and per mg immobilized enzyme (measured by absorption spectra). The UV-vis instrument can accurately measure the enzymatic oxidation rate (Vₒ) up to 0.001 μM min⁻¹ mg⁻¹. The enzymatic oxidation rate of the immobilized tyrosinase on glass beads was 0.02 min⁻¹ mg⁻¹ for p-cresol with 6% water in toluene but not detectable for BPA even with over 6% water in toluene. The limited surface area (around 0.24 m² g⁻¹) of glass beads might result in a low loading efficiency of the tyrosinase.

Mesoporous carbon was chosen as a support to immobilize the tyrosinase due to its large surface area (500-1000 m² g⁻¹). In this experiment, the specific surface area of mesoporous carbon was 582 m² g⁻¹. When 5 mg mL⁻¹ mesoporous carbon was mixed with 50 mmol L⁻¹ p-cresol or 0.2 mmol L⁻¹ BPA in aqueous solution, over 90% BPA and p-cresol were adsorbed from aqueous solution to mesoporous carbon by physical adsorption. However, compared with aqueous solution, less than 5% BPA and p-cresol were adsorbed from toluene to mesoporous carbon. Despite these advantages of mesoporous carbon, FIG. 2, panel A indicated that the reaction product was still not detectable even when 8% of water was added to the toluene. During the adsorption process, it is possible that a certain degree of conformational change in the tyrosinase resulted in the loss of biological activity.

The experiments showed that only mesoporous carbon is not readily biocompatible. Coating polymers onto carbon materials could change its surface properties and create a biocompatible nanocomposite. Polyethylenimine (PEI), a cationic polymer, is used in encapsulating protein antigen and DNA via electrostatic attraction. After PEI was identified as the potential candidate of the coating material, from 1% to 8% of water was added to the toluene to test the immobilized tyrosinase activity. An adequate water content in the reaction medium is crucial. As shown in FIG. 9, panel A, higher water percentages led to greater rates of enzymatic oxidation. After adding 6% water, the rate of enzymatic oxidation leveled off and decreased slightly. After optimizing the water content, the weight ratio between PEI and mesoporous carbon as an enzyme immobilization platform was then investigated. The rate of enzymatic oxidation increased until the weight ratio was 1:1 (PEI:mesoporous carbon) and then decreased with a 2:1 ratio (FIG. 9, panel B). Therefore, the optimized condition include a 1:1 PEI:mesoporous carbon weight ratio for the immobilization platform with 6% of water in the reaction medium.

Physical Characterization of PEI/Mesoporous Carbon and Tyrosinase Immobilization onto PEI/Mesoporous Carbon.

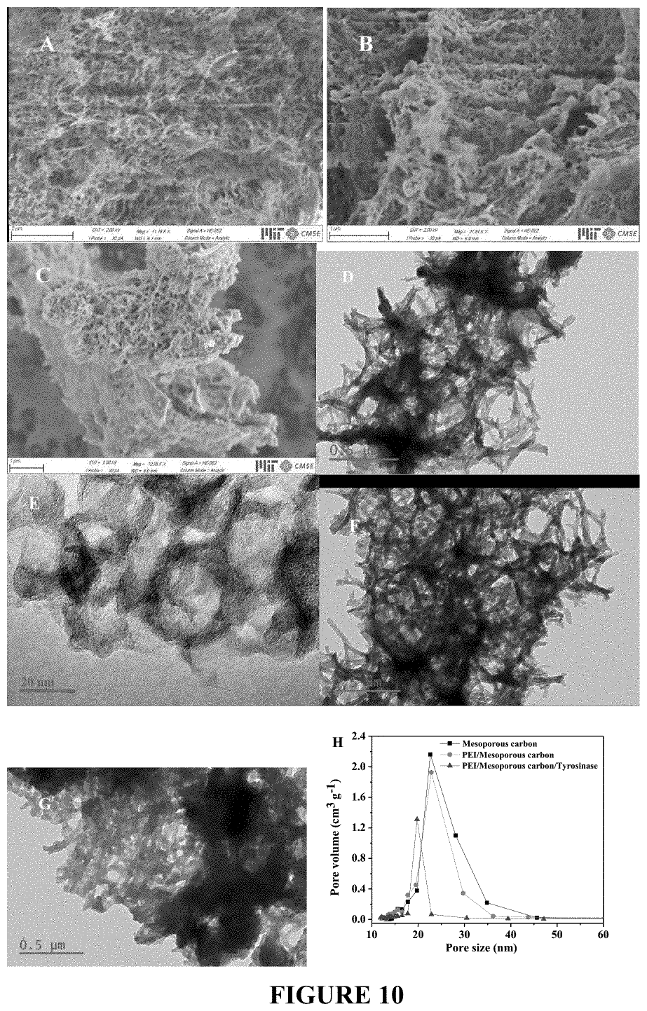

The surface of the PEI coated mesoporous carbon was characterized by SEM, TEM and nitrogen adsorption-desorption isotherms. FIG. 10, panel A demonstrates the ordered pores morphology of unmodified mesoporous carbon. After PEI was coated onto the mesoporous carbon (FIG. 10, panel B), the surface appeared rougher. This alteration supports the conclusion that the PEI had been coated onto the surface of the mesoporous carbon. Then, after immobilization of tyrosinase onto the PEI/mesoporous carbon (FIG. 10, panel C), its surface does not change a lot. The TEM images were used for confirming the SEM results. FIG. 10, panels D and 3E (the enlarged view) showed uniformly distributed spherical pores of mesoporous carbon, and its edge is very clear. After PEI coated onto the mesoporous carbon (FIG. 10, panel F), the edge of the nanocomposite appears rougher. Subsequently, after immobilization of tyrosinase onto the PEI/mesoporous carbon (FIG. 10, panel G), its edges become even rougher than the bare nanocomposite.

FIG. 10, panel H shows the pore size distribution for mesoporous carbon and the nanocomposite by the Barrett-Joyner-Halenda (BJH) method. The average pore size of mesoporous carbon is 22 nm with a specific surface area and pore volume of 582 m² g⁻¹ and 2.1 cm³ respectively. After coating PEI on the mesoporous carbon surface, the pore size remains unchanged while the surface area and pore volume decreased to 505 m² g⁻¹ and 1.9 cm³ g⁻¹, respectively. This suggests that the mesoporous carbon had been coated by PEI which slightly reduced surface area and pore volume. After tyrosinase immobilization onto the PEI coated mesoporous carbon surface, its surface area and pore volume further decreased to 200 m² g⁻¹ and 1.3 cm³ g⁻¹, respectively, indicating that the tyrosinase had been immobilized onto the surface of coated mesoporous carbon.

Coating Materials Influence the Immobilized Tyrosinase Biocatalysis Ability.

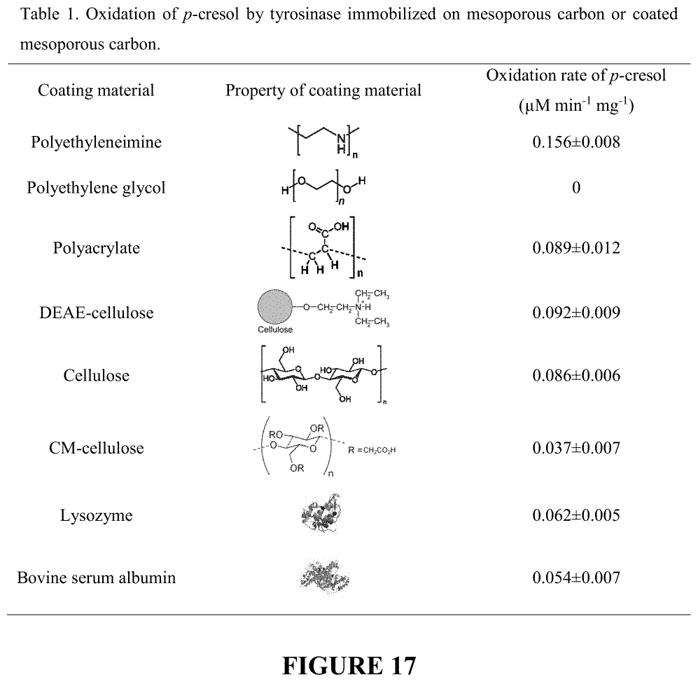

To understand the role played by the PEI coating, several different polymers were also used as coating materials. Table 1 (FIG. 17) showed that the rates of enzymatic oxidation after tyrosinase immobilization onto polyethylene glycol (PEG) coated mesoporous carbon, polyacrylate (PAL) coated mesoporous carbon, di ethylaminoethyl cellulose (DEAE-cellulose) coated mesoporous carbon, cellulose coated mesoporous carbon, carboxymethyl cellulose (CM-cellulose) coated mesoporous carbon, bovine serum albumin (BSA) coated mesoporous carbon and lysozyme coated mesoporous carbon were 0, 0.089, 0.092, 0.086, 0.037, 0.062, and 0.054 μM min⁻¹ mg⁻¹, respectively.

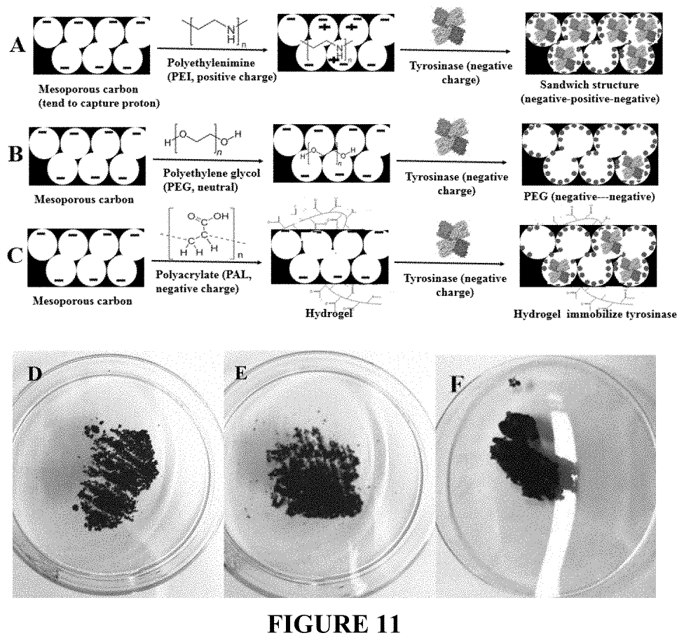

The coating material was divided into three groups for a better understanding. The three traditional polymers can be categorized based on the charges on their surface: PEI (positive charge), PEG (neutral) and PAL (negative charge). Table 1 showed that the rate of enzymatic oxidation after tyrosinase immobilization was highest for the PEI coated mesoporous carbon. The electrostatic attractions likely assist the coating of the positively charged PEI onto the negatively charged mesoporous carbon. The abundant amine groups on PEI (where the positive charges are from) could also electrostatically attract the negative charges of tyrosinase (as shown in Scheme 1). In this way, a high catalytic efficiency of the immobilized tyrosinase could be achieved. PAL, an anionic polymer with negatively charged carboxylic groups in the main chain, had the next highest rate of oxidation. As shown in the photograph of FIG. 11, polyacrylate can bind with mesoporous carbon to form a black hydrogel, even though PAL is a negative charged polymer. The retained water in hydrogel could be beneficial for keeping the enzymatic activity of tyrosinase. However, there was electrostatic repulsion between PAL and the tyrosinase, limiting its catalytic efficiency. PEG, a neutral polymer, provided a steric barrier for avoiding proteins sticking onto the surface of the materials in previous research. After PEG coating onto mesoporous carbon, the catalytic efficiency of the immobilized tyrosinase was still undetectable. This indicates that PEG did not improve the catalytic efficiency of the immobilized tyrosinase.

The second polymer coating group consists of three differently charged cellulose, and their chemical structures were shown in Table 1. Table 1 also showed that the rate of enzymatic oxidation after tyrosinase immobilization was highest for the positively charged DEAE-cellulose, corroborating the results from the first polymer screening. The negatively charged CM-cellulose coated mesoporous carbon had the lowest activity among the cellulose coatings possibly due to electrostatic repulsion. Neutral cellulose coated mesoporous carbon had a similar activity to PAL likely due to its ample hydroxyl groups which could create a similar hydrogen bonding network to maintain the catalytic efficiency of the immobilized tyrosinase. These results indicate that electrostatic attraction plays a main role in immobilizing tyrosinase.

In addition to using synthetic polymers as coating materials, we also used proteins to coat mesoporous carbon as previous reports showed that protein coatings could provide a biocompatible surface for immobilizing enzymes. Two common proteins, lysozyme and bovine serum albumin (BSA), were selected for coatings on the surface of mesoporous carbon. Table 1 shows that the rates of enzymatic oxidation were similar for both lysozyme and BSA. Lysozyme (molecular weight: 14.3 kDa) is thermally stable and its isoelectric point is 11.35. Thus lysozyme likely played a similar role as PEI in creating a layer-by-layer structure of negative and positive charges. BSA (molecular weight: 66.5 kDa) is a serum albumin protein and its isoelectric point is 4.7. It is possible that the positively charged residues on the BSA surface interacted with the surface of mesoporous carbon, allowing the BSA to be stably adsorbed. Because both BSA and lysozyme were coated onto mesoporous carbon by a positively charged subdomain, their ample hydroxyl and amino groups could create hydrogen bonding network to maintain the catalytic efficiency of the immobilized tyrosinase. As a result, the oxidation rates of lysozyme coated mesoporous carbon had a slightly higher activity than that of BSA coated mesoporous carbon. These results corroborate that electrostatic attraction plays a main role in immobilizing tyrosinase.

Oxidation of BPA Using the Immobilized Tyrosinase

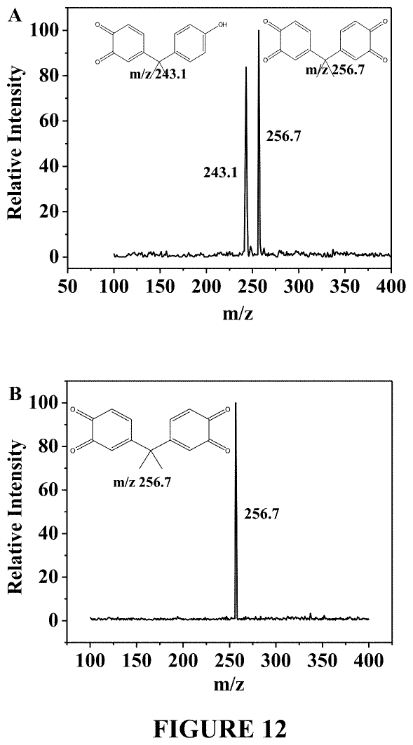

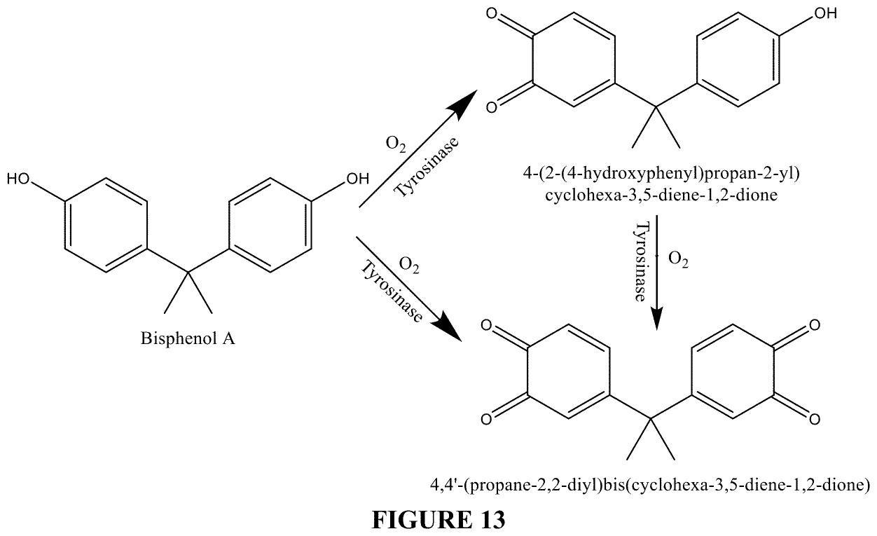

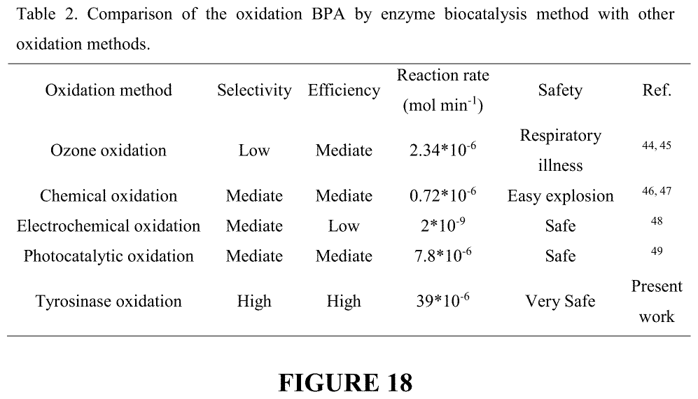

After optimizing the experimental conditions, the tyrosinase immobilization onto PEI/mesoporous carbon was used as a biocatalyst for oxidation of BPA in toluene. The rate of enzymatic oxidation for BPA (10 mmol L⁻¹) in toluene with 6% water was 0.039 μM min′ me. The rate of enzymatic oxidation for BPA is lower than that for p-cresol due to its stronger steric hindrance. As shown in Table 2 (FIG. 18), compared with ozone oxidation of BPA, chemical oxidation of BPA, photocatalytic oxidation of BPA and electrochemical oxidation of BPA, enzyme oxidation of BPA had high selectivity and efficiency. Above all, tyrosinase oxidation of BPA is the safest method among all of them. After 60 min of reaction with the immobilized tyrosinase, BPA was converted into 4-[1-(4-hydroxyphenyl)-1-methyl-ethyl]-1,2-benzoquinone and 4,4′-(1-methylethylidene)bis(1,2-benzoquinone) as oxidation products determined by liquid chromatography-mass spectrometry (FIG. 12). The ratio between the two products was approximately 1:1 (FIG. 12, panel A). After 10 hours of reaction, most of the oxidation product was the bisquinone derivative (FIG. 12, panel B). A potential reaction mechanism is shown in FIG. 13. The o-quinone did not readily form polymers in toluene solution although they are prone to aggregation in aqueous solution.

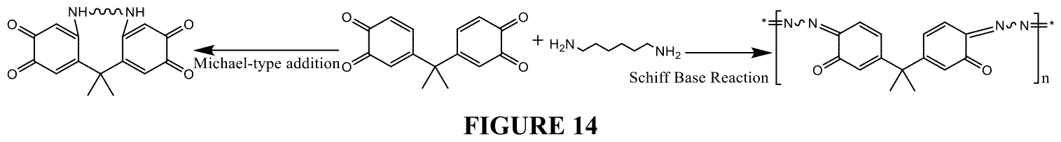

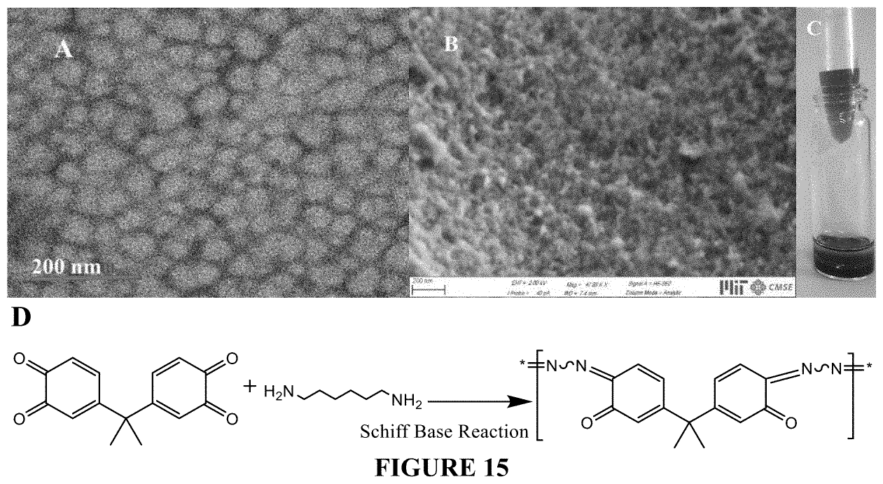

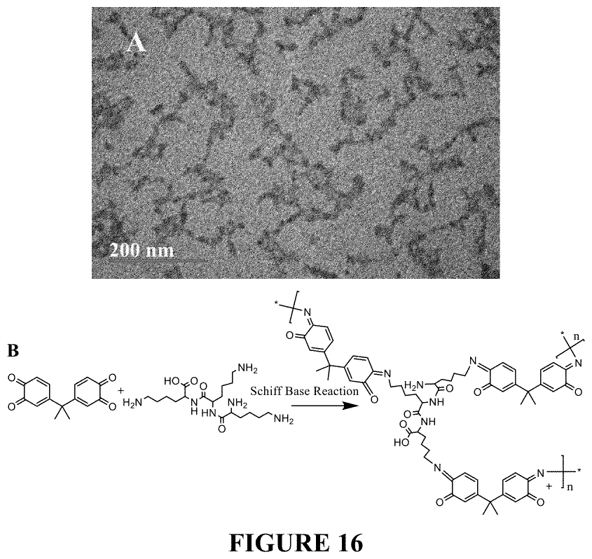

The 4,4′-(1-methylethylidene)bis(1,2-benzoquinone) has four carbonyl groups which may further react with a variety of nucleophiles in various pathways. Well-known nucleophiles, amines may react with the o-quinone to form adducts either by Michael addition or Schiff base reactions, as shown in FIG. 14. Therefore, in this study, 4,4′-(1-methylethylidene)bis(1,2-benzoquinone) and hexamethylenediamine was used to study the crosslinking chemistry, and the reaction mechanism was shown in FIG. 15, panel D. After adding hexamethylenediamine into 4,4′-(1-methylethylidene)bis(1,2-benzoquinone) toluene, the color changed from yellow to purple (FIG. 15, panel C), indicating that a reaction had taken place, and the poly(amino-quinone) polymers whose structure looks like honeycomb were formed (as shown in FIG. 15, panel A). As shown in FIG. 15, panel B, the SEM figure of poly(amino-quinone) polymers demonstrated that there were lots of nanopores on the surface of this polymers, and its surface looks very rough. Already, some of similar polymers have been successfully utilized for adhering metals and alloys as a anticorrosion material. The 4,4′-(1-methylethylidene)bis(1,2-benzoquinone) also could be used for covalent binding peptides (enzymes) and water-insoluble carriers. The main functional groups that take part in the covalent binding of peptides to carrier is a or c-amino group. After trilysine, a short peptide with amino group, reacted with 4,4′-(1-methylethylidene)bis(1,2-benzoquinone) in toluene, a branched polymers were formed (FIG. 16, panel A), and the reaction mechanism was shown in FIG. 16, panel B. In this way, 4,4′-(1-methylethylidene)bis(1,2-benzoquinone) was another condensing reagents like carbodiimide reagents (1-ethyl-3-(3-dimethylaminopropyl) carbodiimide hydrochloride). It can react with carriers containing amino groups with one side quinone group, and the other side quinone group subsequently react with amino groups of the enzyme to produce immobilized enzyme through covalent binding. This bisquinone enriches the immobilization method for proteins or peptides as bi-functional reagents.

This work developed a method to selectively oxidize BPA under mild conditions. The model enzyme (tyrosinase) was directly immobilized onto the surface of mesoporous carbon via physical adsorption ability. The rate of enzymatic oxidation was improved by coating the mesoporous carbon with various polymers. Among these coating materials, PEI resulted in the highest rate of oxidation due to its ability to form a charge sandwich structure to protect tyrosinase. The final oxidation product of BPA is 4,4′-(1-methylethylidene)bis(1,2-benzoquinone), which can be used as a monomer to form the poly(amino-quinone) polymers by Schiff base reactions. This research discusses several polymers which can make mesoporous carbon a promising immobilization platform for applications in biocatalysis, biosensing and drug delivery. This discovery of an effective coating strategy will speed the development of high catalytic efficiency enzyme bioelectronics devices and biofuel battery.

Tyrosinase is a copper-containing enzyme present in plant and animal tissues that catalyzes the production of melanin and other pigments. In organic solvent, tyrosinase can convert N-Acetyl-L-tyrosine ethyl ester (not soluble in aqueous) to a derivative of L-dopamine (a drug used for the treatment of Parkinson's disease). Thus, the performances of tyrosinase in organic solvent have attracted researcher's attention since 1980. In this work, we investigated the thermostable ability of immobilized tyrosinase at high temperature in anhydrous organic solvent. Triethylaminoethyl cellulose (TEAE-Cellulose) was noticed as the best one out of six immobilization platforms. The dry immobilized tyrosinase became extremely thermostable, and it can withstand heating 100° C. for one month in hydrophobic hexane (log p: 4.66, non-polar). Even, in hydrophilic methanol, the immobilized tyrosinase exhibits high activity after incubating 10 min at 100° C., but the immobilized tyrosinase loses its activity instantaneously in aqueous solution. The half-life of the dry immobilized tyrosinase in organic solvent is stronger related with the polarity of the solvent than log P value of organic solvent. This investigation could be valuable for the design of new biocatalysts.

Mimicking tyrosinase has been a longstanding goal of industrial and academic chemists. Tyrosinases (polyphenol oxidase, EC 1.14.18.1), copper-containing oxidoreductases, can enable aerobic oxidation of relatively inert C—H bonds, which remains difficult for synthetic catalysts. Most of the synthetic catalysts can be stable in most of the organic solvents at high temperature (mostly higher than 100° C.). For mimicking tyrosinase, the behavior of tyrosinase in organic solvents need to be explored. The thermostable behavior of the tyrosinase in surrounding solvent is of high significance for mimicking enzymatic catalysis. Tyrosinase is an enzyme with monophenolase and diphenolase activity. Since 1980s, innovative research has shown that tyrosinase is one of enzyme, which can function well in anhydrous media with rarely water, pioneered mainly by Klibanov and co-workers. This research exploded a hot topic in the last century and mushroom tyrosinase has been utilized as an ideal enzyme by lots of workers to develop a consistent general theory to explain the functioning of enzymes in organic media. The interaction between enzyme molecular and the surrounding water in organic media, the effect of the solvent on enzyme regioselectivity and enzymatic catalysis in organic media have been systematically investigated. See, for example, Esguerra, K. V. N.; Lumb, J. P., Selectivity in the Aerobic Dearomatization of Phenols: Total Synthesis of Dehydronornuciferine by Chemo- and Regioselective Oxidation. Angew Chem Int Edit 2018, 57, 1514-1518; Ma, D.; Tu, Z. C.; Wang, H.; Zhang, L.; He, N.; McClements, D. J., Mechanism and kinetics of tyrosinase inhibition by glycolic acid: a study using conventional spectroscopy methods and hydrogen/deuterium exchange coupling with mass spectrometry. Food Funct 2017, 8, 122-131; Ashraf, Z.; Rafiq, M.; Nadeem, H.; Hassan, M.; Afzal, S.; Waseem, M.; Afzal, K.; Latip, J., Carvacrol derivatives as mushroom tyrosinase inhibitors; synthesis, kinetics mechanism and molecular docking studies. Plos One 2017, 12; Rolff, M.; Schottenheim, J.; Decker, H.; Tuczek, F., Copper-O-2 reactivity of tyrosinase models towards external monophenolic substrates: molecular mechanism and comparison with the enzyme. Chem Soc Rev 2011, 40, 4077-4098; Kazandjian, R. Z.; Klibanov, A. M., Regioselective Oxidation of Phenols Catalyzed by Polyphenol Oxidase in Chloroform. J Am Chem Soc 1985, 107, 5448-5450; Klibanov, A. M., Enzyme-Catalyzed Processes in Organic-Solvents. Ann Ny Acad Sci 1987, 501, 129-129; Burton, S. G., Biocatalysis with Polyphenol Oxidase—a Review. Catal Today 1994, 22, 459-487; Oz, F.; Colak, A.; Ozel, A.; Ertunga, N. S.; Sesli, E., Purification and Characterization of a Mushroom Polyphenol Oxidase and Its Activity in Organic Solvents. J Food Biochem 2013, 37, 36-44; Ouml; zel, A.; Colak, A.; Arslan, O.; Yildirim, M., Purification and characterisation of a polyphenol oxidase from Boletus erythropus and investigation of its catalytic efficiency in selected organic solvents. Food Chem 2010, 119, 1044-1049; Zaks, A.; Klibanov, A. M., The Effect of Water on Enzyme Action in Organic Media. J Biol Chem 1988, 263, 8017-8021; and Rubio, E.; Fernandezmayorales, A.; Klibanov, A. M., Effect of the Solvent on Enzyme Regioselectivity. J Am Chem Soc 1991, 113, 695-696, each of which is incorporated by reference in its entirety. But, there is no report available about the thermostable behavior of tyrosinase in different pure organic solvents at high temperature.

To study the thermostable behavior of tyrosinase, the enzyme must be easy to reuse. See, for example, Plothe, R.; Sittko, I.; Lanfer, F.; Fortmann, M.; Roth, M.; Kolbach, V.; Tiller, J. C., Poly(2-ethyloxazoline) as Matrix for Highly Active Electrospun Enzymes in Organic Solvents. Biotechnol Bioeng 2017, 114, 39-45; and Liao, F. S.; Lo, W. S.; Hsu, Y. S.; Wu, C. C.; Wang, S. C.; Shieh, F. K.; Morabito, J. V.; Chou, L. Y.; Wu, K. C. W.; Tsung, C. K., Shielding against Unfolding by Embedding Enzymes in Metal-Organic Frameworks via a de Novo Approach. J Am Chem Soc 2017, 139, 6530-6533, each of which is incorporated by reference in its entirety. In order to overcome this disadvantage, immobilization techniques, which can make the enzyme reuse easier will be the best choice. See, for example, Labus, K.; Turek, A.; Liesiene, J.; Bryjak, J., Efficient Agaricus bisporus tyrosinase immobilization on cellulose-based carriers. Biochem Eng J 2011, 56, 232-240, which is incorporated by reference in its entirety. Through previously reported immobilization methods, the adsorption method provides more active enzyme than the covalent bonding, entrapment, copolymerization and encapsulation methods. See, for example, Jiang, Y. J.; Sun, W. Y.; Wang, Y. P.; Wang, L. H.; Zhou, L. Y.; Gao, J.; He, Y.; Ma, L.; Zhang, X., Protein-based inverse opals: A novel support for enzyme immobilization. Enzyme Microb Tech 2017, 96, 42-46; Patel, S. K. S.; Choi, S. H.; Kang, Y. C.; Lee, J. K., Large-scale aerosol-assisted synthesis of biofriendly Fe2O3 yolk-shell particles: a promising support for enzyme immobilization. Nanoscale 2016, 8, 6728-6738; and Yang, D.; Wang, X. Y.; Shi, J. F.; Wang, X. L.; Zhang, S. H.; Han, P. P.; Jiang, Z. Y., In situ synthesized rGO-Fe3O4 nanocomposites as enzyme immobilization support for achieving high activity recovery and easy recycling. Biochem Eng J 2016, 105, 273-280, each of which is incorporated by reference in its entirety. Among various kinds of supports, polymers are the ideal matrixes because they are of low cost, nontoxic, renewable, biodegradable, and biocompatible. See, Zucca, P.; Fernandez-Lafuente, R.; Sanjust, E., Agarose and Its Derivatives as Supports for Enzyme Immobilization. Molecules 2016, 21, which is incorporated by reference in its entirety. In this research, we systemically compared different polymers as enzyme supports (i.e. cellulose matrixes, Pluronic F68 and Poly(styrene-co-divinylbenzene)) with traditional glass beads as enzyme support.

After optimized the enzyme immobilization material, the organic solvent effect, the position of substituent group and the strength of electron-donating substituent group were investigated to understand how these parameter influenced the immobilized tyrosinase activity. In this work, the immobilized tyrosinase activity was tested in various hydrophilic and hydrophobic organic solvents. After optimizing the organic solvents, three types of substituent positions and different substituent groups of substrate were also tested. Ultimately, the thermostable behavior of the immobilized tyrosinase in organic solvent was systematically assessed. This work provided more reasonable and systematically explanation of the mechanism of tyrosinase in organic solvents which furnished more information for the design of mimicking biocatalyst.

Physical Characterization of TEAE-Cellulose and Tyrosinase Immobilization onto TEAE-Cellulose.

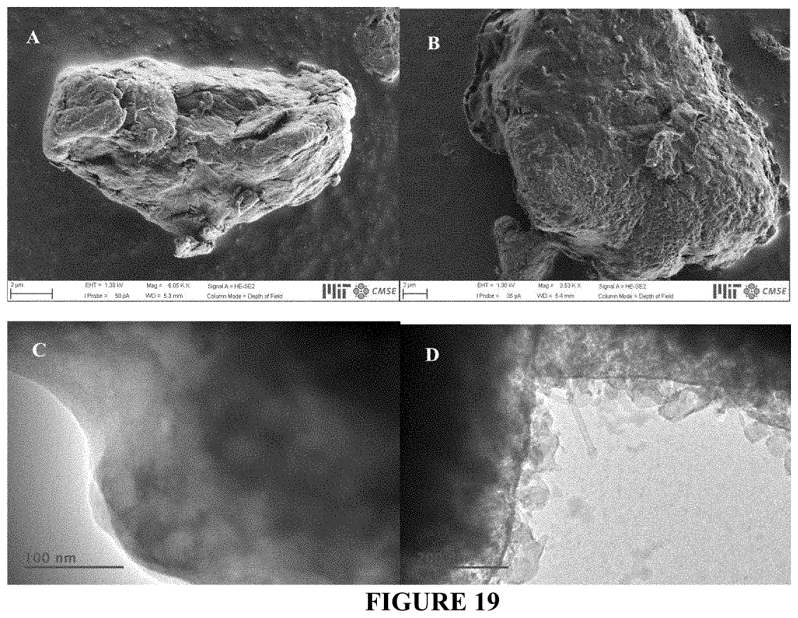

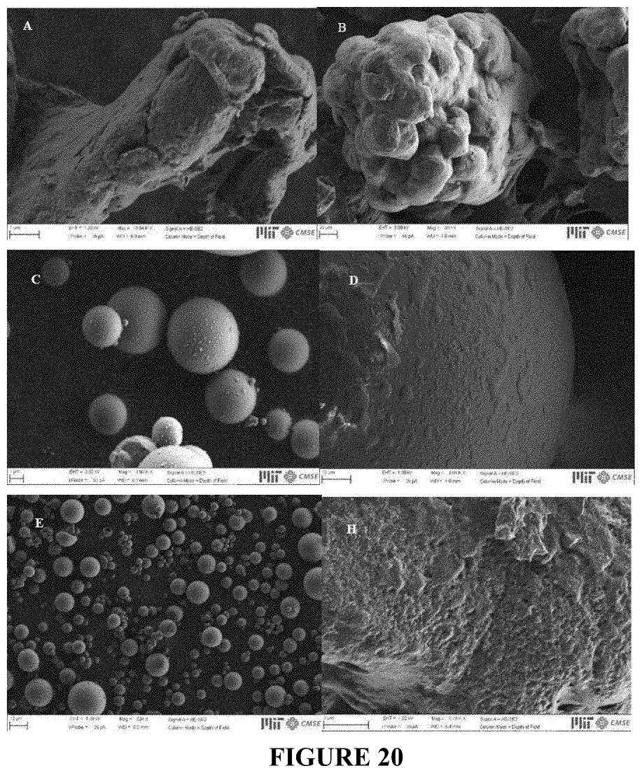

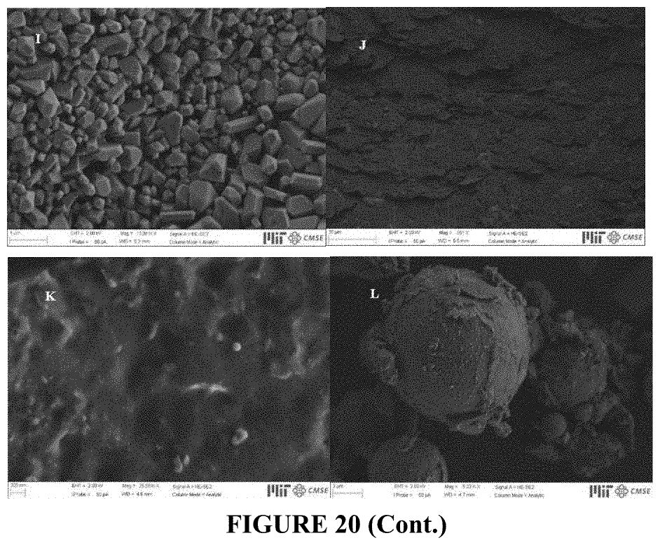

FIG. 19, panel B displayed the SEM image of tyrosinase immobilization onto TEAE-Cellulose as compared to the original TEAE-Cellulose (FIG. 19, panel A). There was apparent difference in the morphology of the original TEAE-Cellulose and tyrosinase immobilization onto TEAE-Cellulose. The original TEAE-Cellulose had a smooth surface, and the TEAE-Cellulose with tyrosinase presented rougher surface and more porosity than the original one. FIG. 19, panel C and FIG. 19, panel D showed the TEM image of original TEAE-Cellulose and TEAE-Cellulose with tyrosinase, respectively. The original TEAE-Cellulose surface was very smooth. After tyrosinase immobilization onto TEAE-Cellulose, its surface showed rougher than the original one. These alterations could be attributed to the tyrosinase immobilization onto the surface of TEAE-Cellulose. FIG. 20 displays the SEM images of A) Cellulose, B) CM-Cellulose, C) Pluronic F68, D) Poly(styrene-co-divinylbenzene) and E) Glass beads. After immobilization tyrosinase onto the surface of materials, Cellulose, CM-Cellulose and Pluronic F68 morphologies totally changed and transform into another morphologies, and all of the materials surface became rough. These alterations could show that the tyrosinase have been immobilized onto the surface of material. After immobilization of tyrosinase, these performs of these materials were assessed by catalysis ability of their immobilized tyrosinase.

### Effects of Immobilization Material on Tyrosinase Biocatalysis Ability.

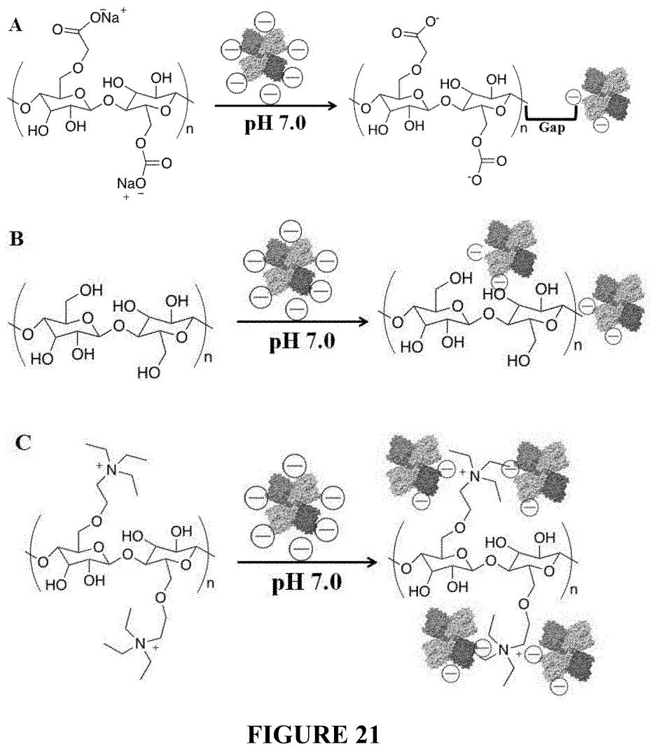

Six kinds of materials were used for the immobilization of tyrosinase: Triethylaminoethyl group of TEAE-Cellulose exhibited positive charge (pH 7.0, PBS solution); cellulose, pluronic F68, poly(styrene-co-divinylbenzene) and glass beads was neutral; and the carboxymethyl group of CM-Cellulose was negative charge. As shown in FIG. 21, the TEAE-Cellulose could bind tyrosinase (negative charge at pH 7.0) much stronger than cellulose and CM-Cellulose because of charge attraction. Due to charge repulsion, CM-Cellulose was not able to maintain a close distance with tyrosinase. While cellulose, pluronic F68, poly(styrene-co-divinylbenzene) and glass beads were physically absorbed tyrosinase onto their surface. The tyrosinase molecular immobilization efficiency of different cellulose may could be arranged as: TEAE-Cellulose>Cellulose=pluronic F68=poly(styrene-co-divinylbenzene)=glass beads>CM-Cellulose.

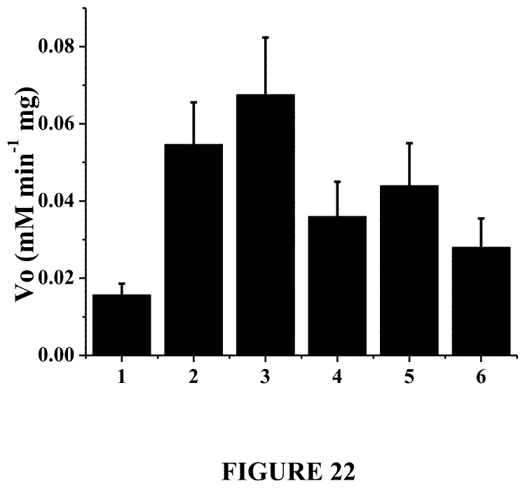

The p-cresol was selected as a test substrate in toluene contained 0.5% (v/v) aqueous buffer (pH 7.0). The tyrosinase can vigorously function in chloroform whereby phenol derivatives can be converted into corresponding stable o-quinones. The enzymatic oxidation product of p-cresol is 4-methyl-1,2-benzoquionone, and its maximum absorbance is 395 nm. Kinetic analysis of tyrosinase using p-cresol substrate in chloroform are shown in FIG. 22. The Vₒ values of tyrosinase molecular immobilization onto TEAE-Cellulose, Cellulose, poly(styrene-co-divinylbenzene), pluronic F68, glass beads and CM-Cellulose were 6.7*10⁻², 5.5*10⁻², 4.4*10⁻², 3.6*10⁻², 2.8*10⁻² and 1.5*10⁻² mmol L⁻¹ min⁻¹, respectively. The Vₒ values of tyrosinase molecular immobilization onto TEAE-Cellulose was the highest among these onto CM-Cellulose, Cellulose, pluronic F68, poly(styrene-co-divinylbenzene) and glass beads, and the Vₒ values of tyrosinase molecular immobilization onto CM-Cellulose was the lowest among these materials. The results were consistent with the mechanism shown in FIG. 21. Due to charge attraction between the tyrosinase molecular and TEAE-Cellulose, the TEAE-Cellulose was displayed as the best supporting platform for tyrosinase and it was chosen as the immobilization material in the following research work.

### Immobilization Enzyme Kinetics in Different Organic Solvent.

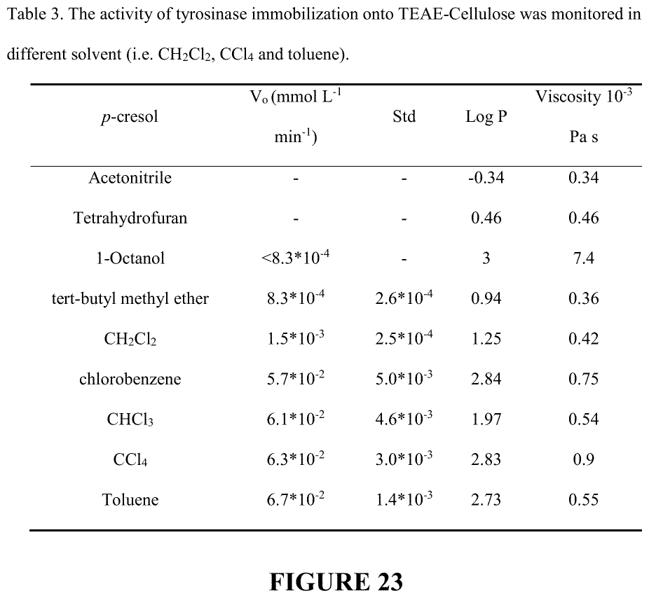

After optimizing tyrosinase immobilization platform, the properties of immobilized tyrosinase was systemically assessed in different organic solvents. Organic solvents can affect the structure of enzymes at the primary, secondary, tertiary and quaternary levels by disrupting interactions, which control the mechanism in the active site, or by altering the thermodynamic nature of the enzyme. It is important to note how organic solvents affect the immobilized tyrosinase activity in organic solvent. From Table 3 (FIG. 23), it demonstrated that the immobilized tyrosinase was completely inactive in hydrophilic solvents viz. acetonitrile (log P, −0.34) and tetrahydrofuran (log P, 0.46). This can be attributed to the fact that the hydrophilic solvents could strip the essential water from tyrosinase's hydration “shell” (hence deactivate the enzyme), even tyrosinase tightly immobilized onto the biocompatible TEAE-Cellulose. On the other hand, the immobilized tyrosinase had oxidation ability in hydrophobic solvent such as tert-butyl methyl ether (log P, 0.94), methylene chloride (log P, 1.25), chloroform (log P, 1.97), carbon tetrachloride (log P, 2.83), chlorobenzene (log P, 2.84) and toluene (log P, 2.73). Octanol-water partition coefficient (log P) is used in QSAR studies and rational chemical design as a measure of molecular hydrophobicity. Our results suggested that the hydrophobicity of organic solvent played an important role in tyrosinase efficiency. As the hydrophobicity of organic solvents increased, most of the biocatalysis efficiency of the immobilized tyrosinase increased. There is an exception to every rule. 1-octanol belong to hydrophobic solvent and the number of log P is relative high (achieved 3, similarly with toluene and carbon tetrachloride), the Vₒ number of the immobilized tyrosinase in 1-octanol is supposed to be high but relative low (less than 8.3*10⁻⁴). In this case, the viscosity of 1-octanol achieved as high as 7.4*10⁻³ Pa*s and mass-transfer rate of substrate in 1-octanol would be very slow, this might be one of the most important parameters in determining the biocatalysis efficiency of the immobilized tyrosinase. In conclusion, toluene was selected the best solvent to test the TEAE-Cellulose immobilized tyrosinase activity.

### The Biocatalysis Efficiency of the Immobilized Tyrosinase for Different Substituent Group Site of Para-Cresol Isomers in Hydrophobic Organic Solvent.

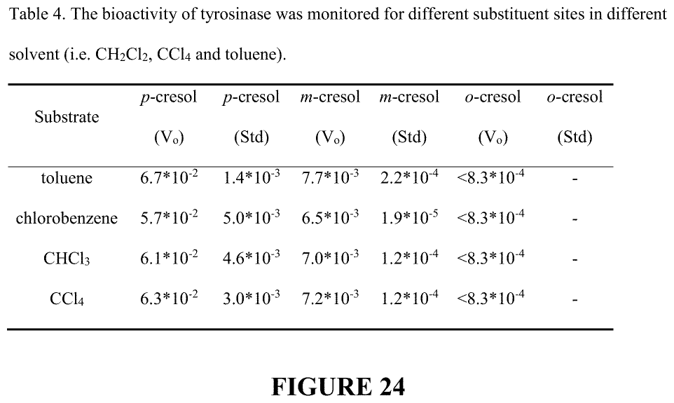

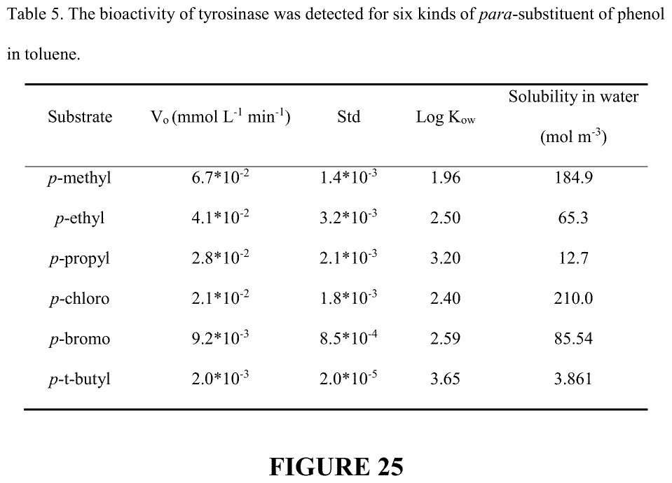

After optimizing the immobilized tyrosinase solvent condition, the relationship between the structure of substrate and the activity of the immobilized tyrosinase was systemically assessed. First, the immobilized tyrosinase was used for assessing how the substitute position (three isomers of p-cresol) influenced the selectivity of the immobilized tyrosinase in toluene. Table 4 (FIG. 24) showed that the Vₒ value for tyrosinase in the toluene medium, using p-cresol as substrate, was 8.7-fold higher than those obtained with m-cresol as substrate and more than 30-fold higher than those with o-cresol as substrate (the Vₒ number was too small). Ortho-substituents group was not obviously detectable because such substrates sterically hinder the accessibility of the phenolic hydroxyl moiety to the active-site of tyrosinase. The sterically influence of para substituent group of phenol for the biocatalysis of tyrosinase was less than that of meta or ortho substituents. It was essential to know how the various para substituents group influence the catalytic efficiencies of the immobilized tyrosinase. Differentpara-substituent groups of phenol were tested in toluene. Table 5 (FIG. 25) showed that the Vₒ value of tyrosinase biocatalysis for para substituent were: p-methyl>p-ethyl>p-propyl>p-chloro>p-bromo>p-t-butyl. The methyl, ethyl, propyl and t-butyl groups were electron-donating group. As increasing the electron-donating ability and sterical size of these para-substituent, the corresponding Vₒ value of the immobilized tyrosinase decreased.

It indicated that the tyrosinase activity was related with the electron-donating ability and sterical size of substitute. Whereas p-chloro and p-bromo were electron-withdrawing group, their corresponding Vₒ value of the immobilized tyrosinase decreased with their deactivating ability. The conclusion was consistent with the quantitative assessment of enzymatic catalysis by Dordick' group.

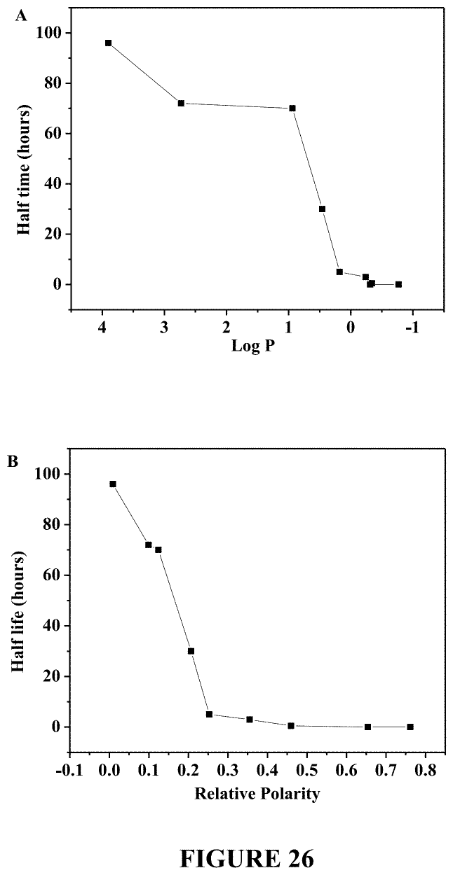

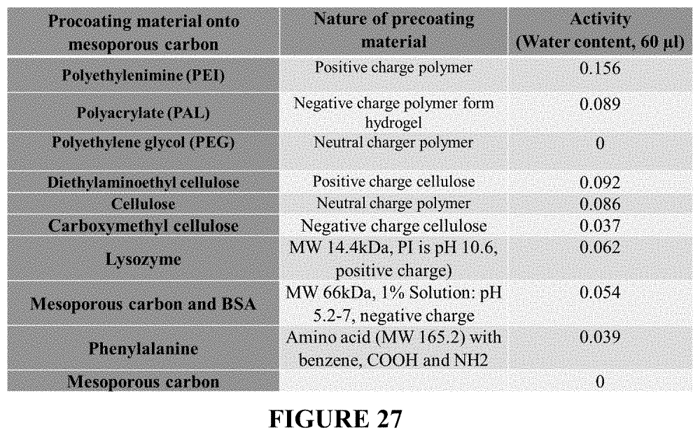

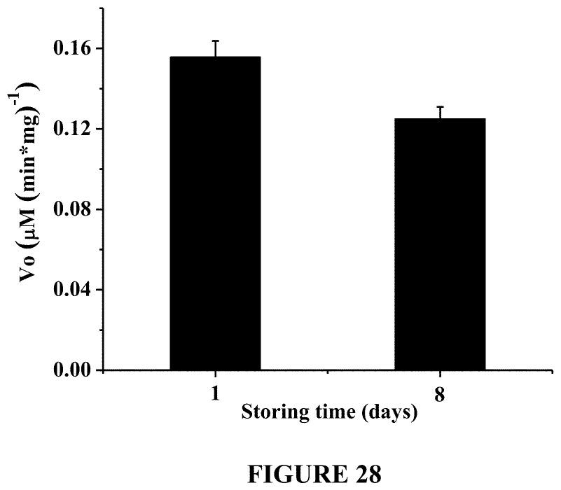

After optimizing the immobilized platform, the substrate and the solvent, the behavior of tyrosinase in anhydrous organic solvent at high temperature need to be explored. In this way, the immobilized tyrosinase was treated as a synthetic catalyst. Nine kinds of anhydrous organic solvents were used for storing the immobilized tyrosinase at 100° C. Nine anhydrous organic solvents are methanol (log P, −0.77; relative polarity, 0.762), ethanol (log P, −0.31; relative polarity, 0.654), acetonitrile (log P, −0.34; relative polarity, 0.46), acetone (log P, −0.24; relative polarity, 0.355), methyl acetate (log P, 0.18; relative polarity, 0.762), tetrahydrafuran (log P, 0.46; relative polarity, 0.207), methyl t-butyl ether (log P, 0.94; relative polarity, 0.124), toluene (log P, 2.73; relative polarity, 0.099) and hexane (log P, 3.9; relative polarity, 0.009). As shown in FIG. 26, the half-life of the immobilized tyrosinase much more related with relative polarity number than log P values. After the immobilized tyrosinase immerses in anhydrous methanol at 100° C. for 10 minutes, the immobilized tyrosinase still keeps 60% activity. But the immobilized tyrosinase loses the activity immediately in water solution at 100° C. This indicated that the immobilized tyrosinase could keep its catalytic structure and be used as a synthetic catalyst in anhydrous organic solvent at high temperature. This research gives lots of information for mimicking enzyme reactions.

Based on certain criteria, triethylaminoethyl cellulose (TEAE-Cellulose) was selected as the best immobilization platform from six kinds of materials. Compared with traditional immobilization material (glass beads), the activity of tyrosinase immobilization onto TEAE-Cellulose was two times higher than that of immobilization onto glass beads. Triethylaminoethyl group of TEAE-Cellulose exhibited positive charge (pH 7.0, PBS solution), the molecular charge interactions between tyrosinase (negative) and TEAE-Cellulose (positive) was stronger than physical absorption interactions between tyrosinase and glass beads (other neutral material). The immobilized tyrosinase showed potential to be a catalyst in organic solvent without water.

Experimental Section

Materials.

BPA, para-cresol (p-cresol) p-chlorophenol, p-methoxyphenol and other chemicals were obtained from Sigma Aldrich (USA) and their purity were higher than 99%. Mushroom Tyrosinase (50 KU) was purchased from Sigma Aldrich (USA) as a solid with a specific activity of 2430 unit/mg (1 unit is defined as the enzyme activity resulting in an increase in absorbance at 280 nm of 0.001 at pH 6.5 at 25° C. in a 3 mL reaction volume containing L-tyrosine). Mesoporous carbon (MPC), Polyethyleneimine (PEI, Mn 1200), polyethylene glycol (PEG, Mn 1500), polyacrylate (PAL, Mn 1250), diethylaminoethyl-cellulose (DEAE-cellulose, microgranular), carboxymethyl-cellulose (CM-cellulose, microgranular), cellulose (fiber), hen egg-white lysozyme (Mw: 14.4 kDa), triethylaminoethyl cellulose (TEAE-Cellulose) and bovine serum albumin (BSA, Mw: 66 kDa) were also from Sigma Aldrich (USA).

Instrumentations.

Scanning electron microscope (SEM) images were obtained by a high-resolution SEM (Zeiss Merlin, Germany) with a resolution of 0.8 nm at 15 KV and 1.4 nm at 1 KV. Transmission electron microscopes (TEM) images were obtained from a FEI Tecnai Multipurpose TEM (ThermoFisher Scientific, USA). Nitrogen adsorption-desorption isotherms were obtained using a Micromeritics ASAP 2010 apparatus. Liquid chromatography-mass spectrometry (LC-MS, Agilent 6000 series, USA) was used to monitor the mass of the product. Absorption spectroscopy was detected by a UV-vis spectrophotometry (ThermoFisher nanodrop 2000c, USA).

Preparation of Tyrosinase and Immobilization onto Mesoporous Carbon/Polyethylenimine Nanocomposite.

Mesoporous carbon can be purchased or prepared by the following method.

Mesoporous carbon was synthesized using 22 nm silica nanospheres as template. The synthesis of monodisperse silica nanospheres (22 nm) was based on the so-called “Stober method” reported by our previous method. See, for example, Wu, L.; Lu, X.; Zhang, H.; Chen, J. ChemSusChem 2012, 5, (10), 1918-1925, which is incorporated by reference in its entirety. In short, tetraethoxysilane (TEOS), L-lysine, and H₂O were mixed together with a weight ratio of 1:0.01:78. The above mixed solution was stirred for 12 hr under 80° C. to obtain 22 nm silica nanospheres. Mesoporous carbon was synthesized using the obtained 22 nm silica nanospheres as template according to the above reported procedure. The procedure as follows: 1.0 g dried silica spheres were impregnated with 0.5 mM Ni(NO₃)₂.6H₂O aqueous solution and then dried at 45° C. After being ground in an agate mortar, these silica particles were pressed into pellets. Then, silica pellets were immersed into the preliminarily polymerized polystyrene, followed by heating the composite at 160° C. for 24 hr to allow the impregnation of polystyrene into the interstices of the silica template. Thereafter, the composite was allowed to undergo pyrolysis/carbonization at 950° C. for 3 hr under nitrogen atmosphere, and cooled to the room temperature. The silica spheres were removed from the composite using 20% HF solution for 24 hr, and drying at 120° C. to yield the final graphitized ordered mesoporous carbon.

To form the PEI coated mesoporous carbon, 20 mL of 5 mg mL⁻¹ mesoporous carbon was mixed with 20 mL of 5 mg mL⁻¹ PEI and stirred at room temperature for 18 hr. Then the sediment was separated by centrifugation at 4,500 rpm for 25 min, washed with deionized water thrice, recovered by decantation, and dried at room temperature for 3 hr to obtain the final PEI/mesoporous carbon nanocomposite.

Solid tyrosinase (2 mg) was dissolved in 0.2 mL of 50 mmol L−1, pH 7.0, and then 10 mg of PEI/mesoporous carbon was added. The sticky mixture was spread on a watch glass and left to dry at room temperature for 2 hr. The tyrosinase immobilization onto other materials was prepared following a similar protocol.

UV-Vis Spectrophotometry Detection Tyrosinase-Catalyzed Oxidation Product.

The time course for tyrosinase-catalyzed oxidation of p-cresol or BPA in organic solvents was monitored using a UV-Vis Nanodrop 2000C spectrophotometer (ThermoFisher Scientific) using the following procedure. 1 mL of 50 mmol L⁻¹p-cresol in an organic solvent was added into a 5 mL round-bottom flask, followed by addition of 5 μL of 50 mmol L⁻¹ phosphate buffer (pH 7.0) and the immobilized tyrosinase, and the suspension was mixed using a magnetic stirrer at 250 rpm and 25° C. Aliquots of the liquid were periodically withdrawn, and their absorption spectra recorded in the range of 400-600 nm. The detection procedure of bisphenol A (50 mmol L⁻¹) in a given solvent was similar protocol as p-cresol in a given solvent.

Preparation of Tyrosinase and Immobilization onto TEAE-Cellulose.

Tyrosinase (6 mg) was dissolved in 0.3 mL of 50 mmol L⁻¹ phosphate buffer (pH 7.0), and then 60 mg of cellulose was added. The sticky mixture was spread on a watch glass and left to dry at room temperature. Tyrosinase immobilization onto CM-Cellulose or other material was prepared following similar protocol as tyrosinase immobilization onto TEAE-Cellulose.

UV-Vis Spectrophotometry Detection Tyrosinase-Catalyzed Oxidation Product.

The time course for tyrosinase-catalyzed oxidation of p-cresol in organic solvents was measured as following procedure. P-cresol was dissolved in a given solvent (its concentration: 50 mmol L⁻¹). 1 mL of p-cresol (50 mmol L⁻¹) in organic solvent was added into 5 mL round-bottom flask, followed by addition of 5 μL 50 mmol L⁻¹ phosphate buffer (pH 7.0). Then the immobilized tyrosinase was added, and the suspension was stirred by magnetic stirrers at 250 rpm and 25° C. Aliquots of the liquid were withdrawn and their absorption spectrum recorded in the range 400-600 nm. The detection procedure of other substrates in a given solvent was similar protocol asp-cresol in a given solvent.

Thermostable Ability Test of Tyrosinase in Organic Solvent at High Temperature.

The immobilized tyrosinase was put into a round bottom flask with anhydrous organic solvent under stirring at 100° C. After 1 hour, the anhydrous organic solvent was removed and added 1 mL of p-cresol with 5 μL 50 mmol L⁻¹ phosphate buffer (pH 7.0). The detection procedure was same as the above protocol.

References, each of which is incorporated by reference in its entirety.
1. Le, H. H.; Carlson, E. M.; Chua, J. P.; Belcher, S. M. Toxicol Lett 2008, 176, (2), 149-156.
2. Patisaul, H. B.; Todd, K. L.; Mickens, J. A.; Adewale, H. B. Neurotoxicology 2009, 30, (3), 350-357.
3. Gear, R. B.; Belcher, S. M. Sci Rep-Uk 2017, 7, (856), 1-8.
4. Chen, S.; Zhang, J.; Chen, Y.; Zhao, S.; Chen, M.; Li, X.; Maitz, M. F.; Wang, J.; Huang, N. ACS Appl Mater Interfaces 2015, 7, (44), 24510-24522.
5. Pham, M. C.; Hubert, S.; Piro, B.; Maurel, F.; Le Dao, H.; Takenouti, H. Synthetic Met 2004, 140, (2-3), 183-197.
6. Liang, Y. L.; Jing, Y.; Gheytani, S.; Lee, K. Y.; Liu, P.; Facchetti, A.; Yao, Y. Nat Mater 2017, 16, (8), 841-848.
7. Navarro-Suarez, A. M.; Carretero-Gonzalez, J.; Rojo, T.; Armand, M. J Mater Chem A 2017, 5, (44), 23292-23298.
8. Arai, G.; Shoji, K.; Yasumori, I. J Electroanal Chem 2006, 591, (1), 1-6.
9. Martin, M.; Orive, A. G.; Lorenzo-Luis, P.; Creus, A. H.; Gonzalez-Mora, J. L.; Salazar, P. Chemphyschem 2014, 15, (17), 3742-3752.
10. Hans Zimmer; David C. Lankin; Horgan, S. W. Chemical Reviews 1970, 71, (2), 229-247.
11. Esguerra, K. V. N.; Lumb, J. P. Angew Chem Int Edit 2018, 57, (6), 1514-1518.
12. Hoffmann, A.; Citek, C.; Binder, S.; Goos, A.; Rubhausen, M.; Troeppner, O.; Ivanovic-Burmazovic, I.; Wasinger, E. C.; Stack, T. D. P.; Herres-Pawlis, S. Angew Chem Int Edit 2013, 52, (20), 5398-5401.
13. Nicolucci, C.; Rossi, S.; Menale, C.; Godjevargova, T.; Ivanov, Y.; Bianco, M.; Mita, L.; Bencivenga, U.; Mita, D. G.; Diano, N. Biodegradation 2011, 22, (3), 673-683.
14. Battino, R.; Rettich, T. R.; Tominaga, T. J Phys Chem Ref Data 1983, 12, (2), 163-178.
15. Sato, T.; Hamada, Y.; Sumikawa, M.; Araki, S.; Yamamoto, H. Ind Eng Chem Res 2014, 53, (49), 19331-19337.
16. Corrales, J.; Kristofco, L. A.; Steele, W. B.; Yates, B. S.; Breed, C. S.; Williams, E. S.; Brooks, B. W. Dose-Response 2015, 13, (3), 1-29.
17. Zellnitz, S.; Redlinger-Pohn, J. D.; Kappl, M.; Schroettner, H.; Urbanetz, N. A. International journal of pharmaceutics 2013, 447, (1-2), 132-138.
18. Attri, P.; Gaur, J.; Choi, S.; Kim, M.; Bhatia, R.; Kumar, N.; Park, J. H.; Cho, A. E.; Choi, E. H.; Lee, W. Sci Rep-Uk 2017, 7, (2636), 1-14.
19.
20. Niesner, R.; Heintz, A. J Chem Eng Data 2000, 45, (6), 1121-1124.
21. Akbar, U.; Aschenbrenner, C. D.; Harper, M. R.; Johnson, H. R.; Dordick, J. S.; Clark, D. S. Biotechnol Bioeng 2007, 96, (6), 1030-1039.
22. Takemori, S.; Furuya, E.; Suzuki, H.; Katagiri, M. Nature 1967, 215, (5099), 417-419.
23. Fang, J.; Wu, K.; Liu, Q. M.; Yang, S. J.; Wen, S. J Porous Mat 2018, 25, (2), 503-509.
24. Shan, C. S.; Yang, H. F.; Han, D. X.; Zhang, Q. X.; Ivaska, A.; Niu, L. Langmuir 2009, 25, (20), 12030-12033.
25. Zhang, S. A.; Yang, K.; Feng, L. Z.; Liu, Z. Carbon 2011, 49, (12), 4040-4049.
26. Lv, J.; Chang, H.; Wang, Y.; Wang, M. M.; Xiao, J. R.; Zhang, Q.; Cheng, Y. Y. J Mater Chem B 2015, 3, (4), 642-650.
27. Sharma, V. K.; Thomas, M.; Klibanov, A. M. Biotechnol Bioeng 2005, 90, (5), 614-620.
28. Zaks, A.; Klibanov, A. M. J Biol Chem 1988, 263, (17), 8017-8021.
29. Hu, Y.; Han, W. F.; Huang, G. H.; Zhou, W. Y.; Yang, Z. H.; Wang, C. Y. Macromol Chem Physic 2016, 217, (24), 2717-2725.
30. Keplinger, C.; Sun, J. Y.; Foo, C. C.; Rothemund, P.; Whitesides, G. M.; Suo, Z. G. Science 2013, 341, (6149), 984-987.
31. Park, J. K.; Seo, S. K.; Cho, S.; Kim, H. S.; Lee, C. H. J Nanosci Nanotechno 2018, 18, (3), 1611-1614.
32. Ma, Y. D.; Dong, J. L.; Bhattacharjee, S.; Wijeratne, S.; Bruening, M. L.; Baker, G. L. Langmuir 2013, 29, (9), 2946-2954.
33. Pelaz, B.; del Pino, P.; Maffre, P.; Hartmann, R.; Gallego, M.; Rivera-Fernandez, S.; de la Fuente, J. M.; Nienhaus, G. U.; Parak, W. J. Acs Nano 2015, 9, (7), 6996-7008.
34. Alcantar, N. A.; Aydil, E. S.; Israelachvili, J. N. J Biomed Mater Res 2000, 51, (3), 343-351.
35. Ung, V. Y. L.; Foshaug, R. R.; MacFarlane, S. M.; Churchill, T. A.; Doyle, J. S. G.; Sydora, B. C.; Fedorak, R. N. Digest Dis Sci 2010, 55, (5), 1272-1277.
36. Arola, S.; Tammelin, T.; Setala, H.; Tullila, A.; Linder, M. B. Biomacromolecules 2012, 13, (3), 594-603.
37. Fu, X. W.; Wang, Y.; Zhong, W. H.; Cao, G. Z. Acs Omega 2017, 2, (4), 1679-1686.
38. Fiegel, V.; Harlepp, S.; Begin-Colin, S.; Begin, D.; Mertz, D. Chemistry 2018, 24, 1-10.
39. Nakamura, S.; Kobayashi, K.; Kato, A. J Agr Food Chem 1994, 42, (12), 2688-2691.
40. Phan, H. T. M.; Bartelt-Hunt, S.; Rodenhausen, K. B.; Schubert, M.; Bartz, J. C. Plos One 2015, 10, (10), 1-20.
41. Kubiak-Ossowska, K.; Jachimska, B.; Mulheran, P. A. J Phys Chem B 2016, 120, (40), 10463-10468.
42. Kazandjian, R. Z.; Klibanov, A. M. J Am Chem Soc 1985, 107, (19), 5448-5450.
43. Han, M. J.; Bie, H. M.; Nikles, D. E.; Warren, G. W. J Polym Sci Pol Chem 2000, 38, (16), 2893-2899.
44. Deborde, M.; Rabouan, S.; Mazellier, P.; Duguet, J. P.; Legube, B. Water Res 2008, 42, (16), 4299-4308.
45. Park, C. G.; Choi, E. S.; Jeon, H. W.; Lee, J. H.; Sung, B. W.; Cho, Y. H.; Ko, K. B. Desalin Water Treat 2014, 52, (4-6), 797-804.
46. Yoshida, M.; Ono, H.; Mori, Y.; Chuda, Y.; Onishi, K. Biosci Biotech Bloch 2001, 65, (6), 1444-1446.
47. Olmez-Hanci, T.; Arslan-Alaton, I.; Genc, B. J Hazard Mater 2013, 263, 283-290.
48. Burgos-Castillo, R. C.; Sires, I.; Sillanpaa, M.; Brillas, E. Chemosphere 2018, 194, 812-820.
49. Ng, J. W.; Wang, X. P.; Sun, D. D. Appl Catal B-Environ 2011, 110, 260-272.

Other embodiments are within the scope of the following claims.

What is claimed is:

1. A composite comprising:
a porous carbon carrier;
a polymer coating on a surface of the porous carbon carrier; and
an enzyme associated with the polymer.

2. The composite of claim 1, wherein the porous carbon carrier includes carbon nanoparticles, carbon black or a mesoporous carbon.

3. The composite of claim 1, wherein the porous carbon carrier includes a mesoporous carbon.

4. The composite of claim 1, wherein the porous carbon carrier has a pore-size distribution of about 10 to 50 nm, 15 to 50 nm or 20 to 25 nm.

5. The composite of claim 1, wherein the porous carbon carrier has a pore-size distribution of about 15 to 50 nm.

6. The composite of claim 1, wherein the porous carbon carrier has a pore-size distribution of about 20 to 25 nm.

7. The composite of claim 1, wherein the porous carbon carrier has a specific surface area of between 300 and 800 m² g⁻¹.

8. The composite of claim 1, wherein the porous carbon carrier has a pore volume of between 1.5 and 2.5 cm³ g⁻¹.

9. The composite of claim 1, wherein the polymer includes a plurality of amino groups.

10. The composite of claim 1, wherein the polymer includes polyethyleneimine, a polyethylene glycol, a polyacrylate, triethylaminoethyl cellulose, diethylaminoethyl cellulose, cellulose, carboxymethyl cellulose, bovine serum albumin (BSA), or a lysozyme.

11. The composite of claim 1, wherein the polymer includes polyethyleneimine or a triethylaminoethyl cellulose.

12. The composite of claim 1, wherein the enzyme is a tyrosinase.

13. A method of oxidizing a phenol comprising:
suspending a composite including a porous carbon carrier, a polymer coating on a surface of the porous carbon carrier, and an enzyme associated with the polymer in an organic solvent; and
exposing the composite to a phenol in the organic solvent.

14. The method of claim 13, wherein the enzyme is a tyrosinase.

15. The method of claim 13, wherein the polymer includes a plurality of amino groups.

16. The method of claim 13, wherein the polymer includes polyethyleneimine or a triethylaminoethyl cellulose.

17. The method of claim 13, wherein the porous carbon carrier includes a mesoporous carbon.

18. The method of claim 13, wherein the organic solvent includes toluene.

19. The method of claim 13, wherein the phenol includes bisphenol A.

20. The method of claim 13, further comprising exposing the oxidized phenol to a polyamine to form a polymer.
